SEQUENCE LISTING (19 sequences)

SEQ ID NO: 1 - DNA, 24 bp, synthetic construct
  source 1..24: mol_type = other DNA
ttcaagccttctgcctttccaccc

SEQ ID NO: 2 - DNA, 24 bp, synthetic construct
  source 1..24: mol_type = other DNA
tctcagcagcagccacaactccct

SEQ ID NO: 3 - DNA, 21 bp, synthetic construct
  source 1..21: mol_type = other DNA
catgtacgttgctatccaggc

SEQ ID NO: 4 - DNA, 21 bp, synthetic construct
  source 1..21: mol_type = other DNA
ctccttaatgtcacgcacgat

SEQ ID NO: 5 - DNA, 20 bp, synthetic construct
  source 1..20: mol_type = other DNA
gtaacccgttgaaccccatt

SEQ ID NO: 6 - DNA, 20 bp, synthetic construct
  source 1..20: mol_type = other DNA
ccatccaatcggtagtagcg

SEQ ID NO: 7 - DNA, 20 bp, synthetic construct
  source 1..20: mol_type = other DNA
aggaaccgagtctccagtga

SEQ ID NO: 8 - DNA, 20 bp, synthetic construct
  source 1..20: mol_type = other DNA
atcaactatgatcaccgagt

SEQ ID NO: 9 - DNA, 22 bp, synthetic construct
  source 1..22: mol_type = other DNA
tggccaacatgattagtgaacc

SEQ ID NO: 10 - DNA, 21 bp, synthetic construct
  source 1..21: mol_type = other DNA
aagatggcatgagagcatcgt

SEQ ID NO: 11 - DNA, 20 bp, synthetic construct
  source 1..20: mol_type = other DNA
actgatgaagctgcagaacc

SEQ ID NO: 12 - DNA, 20 bp, synthetic construct
  source 1..20: mol_type = other DNA
gtcacccaattctttgatgg

SEQ ID NO: 13 - DNA, 22 bp, synthetic construct
  source 1..22: mol_type = other DNA
gccatgaagcgctacgccctgg

SEQ ID NO: 14 - DNA, 24 bp, synthetic construct
  source 1..24: mol_type = other DNA
tcttgctcacgaatacgacggtgg

SEQ ID NO: 15 - RNA, 20 nt, synthetic construct
  source 1..20: mol_type = other RNA
ctgggagtaaaattcattag

SEQ ID NO: 16 - RNA, 21 nt, synthetic construct
  source 1..21: mol_type = other RNA
ctggtgagtaaaattcattag

SEQ ID NO: 17 - RNA, 47 nt, synthetic construct
  source 1..47: mol_type = other RNA
ctgggagtggccattcgacgacagtgtggtgtaaaggaattcattag

SEQ ID NO: 18 - RNA, 53 nt, synthetic construct
  source 1..53: mol_type = other RNA
ctggtgagtggccattcgacgccacagtgtggtgtcgaatggccattcattag

SEQ ID NO: 19 - RNA, 53 nt, synthetic construct
  source 1..53: mol_type = other RNA
actgggagtggccattcgacgacagtgtggtgtaaaggaattcattagccatg

(19) United States
(12) Patent Application Publication
(10) Pub. No.: US 2026/0325655 A1
(43) Pub. Date: Oct. 22, 2026

(54) DECREASING THE INTRINSICALLY DISORDERED PROTEIN ALPHA-SYNUCLEIN’S LEVELS BY TARGETING ITS STRUCTURED MRNA WITH A RIBONUCLEASE TARGETING CHIMERA

(71) Applicant: University of Florida Research Foundation, Incorporated, Gainesville, FL (US)

(72) Inventors: Matthew D. Disney, Gainesville, FL (US); Jessica L. Childs-Disney, Gainesville, FL (US)

(73) Assignee: University of Florida Research Foundation, Incorporated, Gainesville, FL (US)

(21) Appl. No.: 19/483,334

(22) PCT Filed: May 10, 2024
(86) PCT No.: PCT/US2024/028891

§ 371 Date: Nov. 11, 2025

Related U.S. Application Data
(60) Provisional application No. 63/502,057, filed on May 12, 2023.

(51) Int. Cl. A61K 47/55; A61K 47/54; A61P 25/16; C07D 401/14; C07D 409/14; G01N 33/50

(52) U.S. Cl. CPC A61K 47/55; A61K 47/545; A61P 25/16; C07D 401/14; C07D 409/14; G01N 33/502

(57) ABSTRACT

The present disclosure provides compounds of the formulae herein (e.g., Formula (I)), and pharmaceutically acceptable salts, solvates, hydrates, polymorphs, co-crystals, tautomers, stereoisomers, isotopically labeled compounds, and prodrugs thereof, which bind and/or effect degradation of an RNA target. The present disclosure also provides pharmaceutical compositions and kits comprising the compounds, or pharmaceutically acceptable salts, solvates, hydrates, polymorphs, co-crystals, tautomers, stereoisomers, isotopically labeled compounds, or prodrugs thereof, and methods of treating or preventing diseases by administering to a subject in need thereof the compounds, or pharmaceutically acceptable salts, solvates, hydrates, polymorphs, co-crystals, tautomers, stereoisomers, isotopically labeled compounds, or prodrugs thereof, or pharmaceutical compositions thereof.

### CROSS-REFERENCE TO RELATED APPLICATIONS

This application claims the benefit of and priority under 35 U.S.C. § 119 (e) to U.S. Provisional Application No. 63/502,057, filed May 12, 2023, titled DECREASING THE INTRINSICALLY DISORDERED PROTEIN ALPHA-SYNUCLEIN'S LEVELS BY TARGETING ITS STRUCTURED MRNA WITH A RIBONUCLEASE TARGETING CHIMERA, the contents of which are incorporated herewith by reference in their entirety.

### GOVERNMENT SUPPORT

This invention was made with government support under grant numbers R35 NS116846, R01 GM097455 and NS096898, awarded by the National Institutes of Health. The government has certain rights in the invention.

### REFERENCE TO AN ELECTRONIC SEQUENCE LISTING

The contents of the electronic sequence listing (U120270137WO00-SEQ-JDH.xml; Size: 18,006 bytes; and Date of Creation: May 10, 2024) is herein incorporated by reference in its entirety.

### BACKGROUND OF THE INVENTION

Genome-wide analyses suggest that only 15% of proteins are in druggable families.^{1,2} A goal of chemical biology and medicinal chemistry therefore is to expand the druggable space by developing new approaches to target “undruggable” proteins that typically do not have architectures that are compatible with small molecule binding. Current strategies to expand drugability include the use of proteolysis-targeting chimeras (ProTACs) to degrade protein targets³⁻⁷ and of covalent chemistry to define ligands that bind to shallow pockets in, for example, mutant KRAS,⁸⁻¹¹ and others.^{12,13}

The protein α-synuclein is central to the pathogenesis of Parkinson's disease (PD) and other α-synucleinopathies, as it misfolds, oligomerizes, and forms fibrils.¹⁴ These fibrils can propagate across neurons, aggregate in Lewy bodies and Lewy neurites, and are associated with neuronal degeneration (FIG. 1).¹⁵ A major factor that promotes α-synuclein fibrillization is its concentration, as individuals with multiplication of the SNCA gene locus develop dominantly inherited PD with a gene dosage effect.¹⁶ Thus, reducing the levels of α-synuclein protein is a potential disease-modifying strategy.¹⁷ However, as an intrinsically disordered protein (IDP) that lacks pockets that can typically be bound by small molecules, α-synuclein protein is considered “undruggable”.^{18,19}

One strategy to expand protein druggability, particularly for disease-causing proteins that are overexpressed, is to target their coding mRNAs and inhibit translation.^{20,21} Such an approach could be accomplished by defining structured regions in an mRNA as potential small-molecule binding pockets, followed by identification of lead small molecules that bind these structures. Previously, a small molecule named Synucleozid-1.0²² was designed, which selectively targets the iron-responsive element (IRE) in the 5′ untranslated region (5′ UTR) of SNCA mRNA,²³ which encodes α-synuclein (FIG. 1). The IRE is bound and stabilized by iron regulatory proteins (IRPs) at low iron concentrations, leading to repression of SNCA mRNA translation.²⁴ At higher concentrations of iron, the IRPs are bound to iron rather than the SNCA IRE, increasing the accessibility of the mRNA to the translational machinery. The small molecule Synucleozid-1.0 stabilizes the SNCA IRE structure and is a functional surrogate for the cellularly expressed IRP that regulates the amount of SNCA mRNA loaded into polysomes. However, Synucleozid-1.0 did not have ideal physicochemical properties for CNS penetration.²⁵

### SUMMARY OF THE INVENTION

Another molecule was later discovered with improved potency and selectively and drug-like physicochemical properties named Synucleozid 2.0. To further enhance the potency, Synucleozid 2.0 was converted into an RNA degrader by tethering with a ribonuclease recruiting module, affording a ribonuclease targeting chimera (RiboTAC). Named Syn-RIBOTAC, the degrader molecule's cytoprotective effect was 5-fold greater than the binding small molecule. Transcriptome- and proteome-wide studies confirmed that both compounds selectively target SNCA mRNA in cells and reduced α-synuclein expression levels in PD patient-derived iPSC-induced dopaminergic neurons. Accordingly, in one aspect, the present disclosure provides compounds of Formula (I):

B-L-R (I),

or pharmaceutically acceptable salts, solvates, hydrates, polymorphs, co-crystals, tautomers, stereoisomers, isotopically labeled compounds, or prodrugs thereof, wherein:
B is an RNA binder of Formula (II):

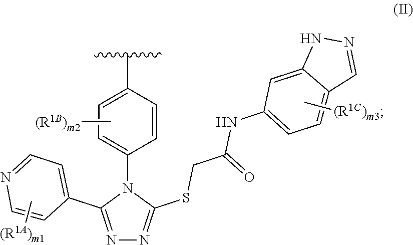

L is a linker;
R is an RNase L recruiter; and
R^{1A}, R^{1B}, R^{1C}, m1, m2, and m3 are as defined herein.

In another aspect, the present disclosure provides pharmaceutical compositions comprising a compound disclosed herein. In some embodiments, the pharmaceutical composition comprises an excipient.

In another aspect, the present disclosure provides methods of binding RNase L in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, binding RNase L comprises activating RNase L. In certain embodiments, binding RNase L comprises inducing RNase L dimerization.

In another aspect, the present disclosure provides methods of binding or effecting degradation of an RNA target in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, the method comprises decreasing an amount of the RNA target. In certain embodiments, the method comprises cleaving the RNA target. In certain embodiments, the RNA target is SNCA mRNA. In certain embodiments, the method further comprises binding an RNA target. In certain embodiments, the method further comprises effecting degradation of an RNA target. In certain embodiments, the method further comprises decreasing an amount of hexokinase 2 (HK2). In certain embodiments, the method further comprises decreasing an amount of α-synuclein.

In another aspect, the present disclosure provides methods of treating or preventing a disease in a subject in need thereof, comprising administering to the subject in need thereof a provided compound or pharmaceutical composition. In certain embodiments, the disease is associated with SNCA mRNA (e.g., a neurodegenerative disease (e.g., Parkinson's Disease, Alzheimer's disease, Lewy bodies' disease, Muscular System Atrophy)). In certain embodiments, the disease is Parkinson's Disease.

In another aspect, the present disclosure provides methods of preparing a compound of Formulae (I-a) or (I-b):

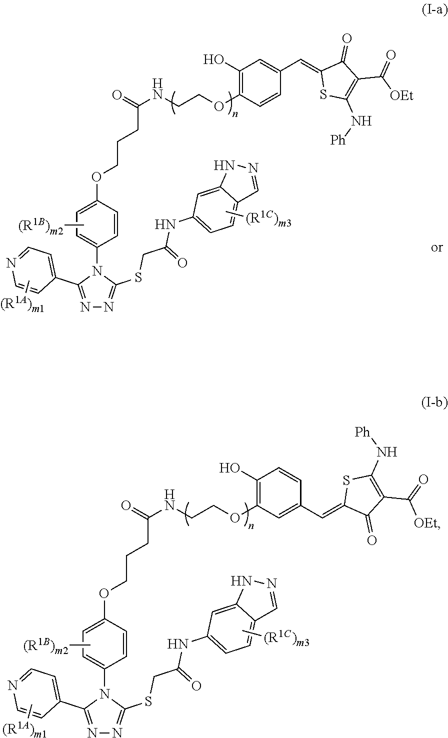

or a pharmaceutically acceptable salt, solvate, hydrate, polymorph, co-crystal, tautomer, stereoisomer, isotopically labeled compound, or prodrug thereof, wherein R^{1A}, R^{1B}, R^{1C}, m1, m2, m3, and n are as defined herein.

In another aspect, the present disclosure provides kits comprising a provided compound or pharmaceutical composition disclosed herein and instructions for its use.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

FIG. 1 shows a schematic depiction of α-synuclein-mediated disease pathway showing small molecules targeting the SNCA IRE can inhibit α-synuclein translation. α-Synuclein can form fibrils that propagate across neurons in the brain and accumulate in Lewy bodies that are associated with neuronal cell death. The IRE harbored in the 5′ UTR of SNCA mRNA regulates α-synuclein translation (SEQ ID NO: 19). Synucleozid-1.0 and Synucleozid-2.0 bind the IRE's A bulge and inhibits α-synuclein translation.²²

FIGS. 2A-2F show Syn-RiboTAC selectively degrades SNCA mRNA in cells. FIG. 2A shows chemical structures of Syn-RiboTAC, and the control compound, Syn-CTRL, which has the recruiter module that is 20-fold less active. The scheme of Syn-RiboTAC recruiting RNase L and cleaves SNCA mRNA in cells is shown at bottom. FIG. 2B shows Syn-RiboTAC and Sy-CTRL inhibit SNCA-UTR-Luciferase signals, while there is no effect on the control luciferase signal that lacks the SNCA-UTR sequence (n=3 biological replicates). FIG. 2C shows Syn-RiboTAC decreases the luciferase mRNA level for SNCA-UTR-Luciferase, while Syn-CTR has no effect on the luciferase mRNA level (n=4 biological replicates). FIG. 2D shows Syn-RiboTAC selectively cleaves SNCA mRNA in SH-SY5Y cells dose-dependently, with no effect on mRNAs of H-Ferritin, APP, and TfR (n=3 biological replicates). FIG. 2E shows Syn-RiboTAC inhibits α-synuclein level in SH-SY5Y cells in a dose dependent manner (n=3 biological replicates). FIG. 2F shows that Syn-RiboTAC showed no effect on the protein levels of H-Ferritin, APP, and TfR in SH-SY5Y cells (n=4 biological replicates). *, p<0.05; **, p<0.01; ***, p<0.001, as determined by two-tailed Student t test. Error bars indicate SD.

FIGS. 3A-3F show Synucleozid-2.0 and Syn-RiboTAC are selective on the proteome and reduced α-synuclein protein abundance in PD patient-derived neurons. FIG. 3A shows global proteomics for SH-SY5Y cells treated by Synucleozid-1.0 (1.5 μM), Synucleozid-2.0 (2 μM), and Syn-RIBOTAC(2 μM). Dotted lines indicate FDR of 1%. Among 2,813 proteins detected, Synucleozid-2.0 affected 150 (0.53%) proteins with 55 proteins downregulated and 95 proteins upregulated. Syn-RiboTAC showed similar selectivity by affecting 194 (0.56%) proteins among 3436 total detected, with 114 proteins downregulated and 80 upregulated. FIG. 3B shows the protein expression levels detectable by proteomics (shown on y-axis) of 7 genes enriched by Syn-CLIP (shown on x-axis) are not affected by the treatment of Synucleozid-2.0 (2 μM). FIG. 3C shows the protein expression levels detectable by proteomics of 7 genes enriched by Syn-CLIP are not affected by the treatment of Syn-RiboTAC(2 μM). FIG. 3D shows Synucleozid-2.0 and Syn-RiboTAC decreased α-synuclein protein levels in iPSC-induced dopaminergic neurons as determined by ELISA assay (n=3 biological replicates). FIG. 3E shows Syn-RiboTAC decreased the SNCA mRNA levels while Synucleozid-2.0 had no effect on SNCA mRNA levels in iPSC-induced dopaminergic neurons (n=3 biological replicates). FIG. 3F shows Syn-RiboTAC improved half of the genes abnormally expressed in patient-derived iPSC induced dopaminergic neurons by converging them towards the levels observed in healthy neurons. *, p<0.05; **, p<0.01; ***, p<0.001, as determined by two-tailed Student t test. Error bars indicate SD.

FIG. 4 shows in vitro binding of Synucleozid-2.0 and its derivatives evaluated by microscale thermophoresis (MST). All compounds showed similar affinities towards the target A bulge model RNA while no saturable binding towards the fully base-paired control construct. Error is the SD from two independent experiments.

FIGS. 5A-5J show Syn-RiboTAC showed improved cytoprotectivity than Synucleozid-2.0 and retained its selectivity on the transcriptome. FIG. 5A shows Syn-RiboTAC(2 μM) protects SH-SY5Y cells against PFFs to a greater extent than Synucleozid 2.0 (10 μM), at a 5-fold lower concentration (n=4 biological replicates). FIG. 5B shows Syn-RiboTAC(2 μM) retains activity as assayed by the SNCA IRE-luciferase reporter 16 h after compound removal while Synucleozid-2.0 (2 μM) does not (n=3 biological replicates). FIG. 5C shows an siRNA pool targeted against RNase L knocks down transcript abundance in SH-SY5Y cells, as determined by RT-qPCR (n=3 biological replicates). FIG. 5D shows the cleavage of SNCA mRNA by Syn-RiboTAC was ablated by siRNA knock-down of RNase L levels. No significant effect on activity was observed when SH-SY5Y cells were transfected with a scrambled control siRNA (n=3 biological replicates). FIG. 5E shows Synucleozid-2.0 competes for the same binding site as Syn-RiboTAC and dose-dependently diminishes the cleavage of Syn-RiboTAC in SH-SY5Y cells (n=3 biological replicates). FIG. 5F shows RNA-seq analysis of SH-SY5Y cells treated with Syn-RiboTAC(2 μM). Among 30,175 genes detected, only 48 (0.16%) were significantly affected (p<0.05 with FDR=1%), with 34 genes downregulated and 14 genes upregulated. FIG. 5G shows RNA-seq analysis of SH-SY5Y cells treated with SNCA siRNA (0.1 μM). FIG. 5H shows transcriptome profiles showed strong correlation between SH-SY5Y cells treated with Syn-RIBOTAC(2 μM) and siRNA (0.1 μM) with R²>0.99. FIG. 5I shows that of these 107 transcripts enriched by Syn-ChemCLIP, 38 were detected in the RNA-seq analysis of Syn-RiboTAC, with only 4 (including SNCA) showing reduced levels, i.e., cleavage. FIG. 5J shows RNA-seq analysis of Syn-RiboTAC(2 μM) in dopaminergic neurons differentiated from iPSCs from the healthy donor (left) and from a PD patient with SNCA triplication (right). *, p<0.05; **, p<0.01; ***, p<0.001, as determined by two-tailed Student t test. Error bars indicate SD.

FIG. 6 shows Syn-RiboTAC modestly reduced HHIPL1 and MYO15B protein levels. SH-SY5Y cells were treated with Syn-RIBOTAC(2 μM) for 48 h, and the protein levels for each protein were measured by Western blot (n=3 biological replicates). *, p<0.05, as determined by two-tailed Student t test. Error bars indicate SD.

FIG. 7 shows Western blot validation of proteomic results for genes enriched by Syn-CLIP. SH-SY5Y cells were treated with Synucleozid 2.0 (2 μM) or Syn-RIBOTAC(2 μM) for 48 h, and the protein levels for each protein were measured by Western blot (n=3 biological replicates). Error bars indicate SD.

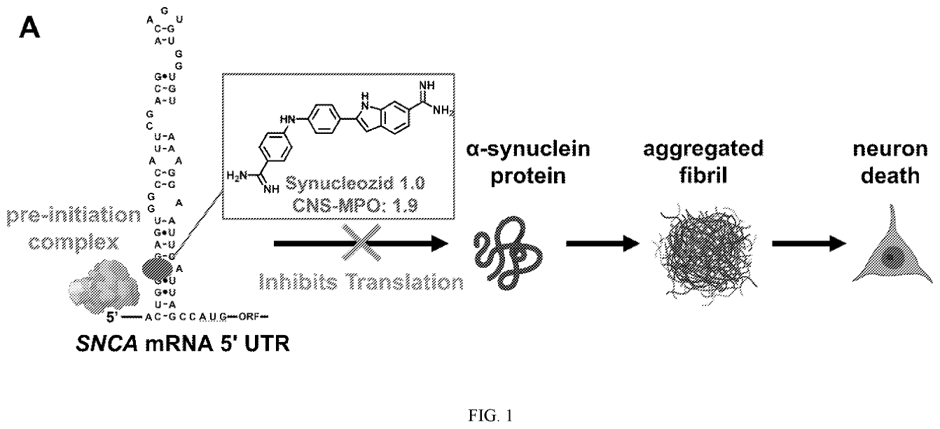

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers. The term “isomers” is intended to include diastereoisomers, enantiomers, regioisomers, structural isomers, rotational isomers, tautomers, and the like. All such isomers of such compounds herein are expressly included in the present invention.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (Cl), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include-CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 2 to 20 carbon atoms and one or more carbon-carbon double bonds (e.g., 1, 2, 3, or 4 double bonds). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₂₀ alkenyl. In certain embodiments, the alkenyl group is a substituted C₂₋₂₀ alkenyl. In an alkenyl group, a C═C double bond for which the stereochemistry is not specified

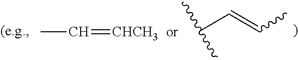

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 11 carbon atoms, at least one double bond, and 1 or more heteroatoms within the parent chain (“heteroC₂₋₁₁ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 carbon atoms, at least one double bond, and 1 heteroatom within the parent chain (“heteroC₂ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₂₀ alkenyl. In certain embodiments, the heteroalkenyl group is a substituted heteroC₂₋₂₀ alkenyl.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₁₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C_{2,7} alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 carbon atoms, at least one triple bond, and 1 heteroatom within the parent chain (“heteroC₂ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₂₀ alkynyl. In certain embodiments, the heteroalkynyl group is a substituted heteroC₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (Cn), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo [3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR)₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}C(═O)₂R^{aa}, ═NR^{bb}, or ═NOR^{cc};
wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂+X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂ (C₁₋₆ alkyl), —OC(═O)(C₁₋₆ alkyl), —OCO₂ (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂ (C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(—O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂.

In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb}) 3 and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula-C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S) S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono-or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphino” refers to the group —P(R^{cc})₂, wherein R^{cc} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl) carbamoyl]trityl Ether (IETr-OR), 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb}) 2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂ (CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺ (C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+ (C₁₋₄ alkyl) 4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound is complexed with the acid in a way that proton transfer (e.g., a complete proton transfer) from the acid to a compound easily occurs at room temperature. In the co-crystal, however, a compound is complexed with the acid in a way that proton transfer from the acid to a herein does not easily occur at room temperature. In certain embodiments, in the co-crystal, there is substantially no proton transfer from the acid to a compound. In certain embodiments, in the co-crystal, there is partial proton transfer from the acid to a compound. Co-crystals may be useful to improve the properties (e.g., solubility, stability, and ease of formulation) of a compound.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers,” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “isotopically labeled compound” refers to a derivative of a compound that only structurally differs from the compound in that at least one atom of the derivative includes at least one isotope enriched above (e.g., enriched 3-, 10-, 30-, 100-, 300-, 1,000-, 3,000-or 10,000-fold above) its natural abundance, whereas each atom of the compound includes isotopes at their natural abundances. In certain embodiments, the isotope enriched above its natural abundance is ²H. In certain embodiments, the isotope enriched above its natural abundance is ¹³C, ¹⁵N, or ¹⁸O.

The term “prodrugs” refers to compounds that have cleavable groups and become by solvolysis or under physiological conditions the compounds described herein, which are pharmaceutically active in vivo. Such examples include choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include, for example, esters prepared by reaction of the parent acid with a suitable alcohol, or amides prepared by reaction of the parent acid compound with a substituted or unsubstituted amine, or acid anhydrides, or mixed anhydrides.

The terms “pharmaceutical composition,” “composition,” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a pharmaceutical composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In some embodiments, the subject is at risk of developing a disease or condition due to environmental factors (e.g., exposure to the sun).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a disease or disorder associated with associated with an RNA target in a subject in need thereof. In certain embodiments, a therapeutically effective amount is an amount sufficient for treating a disease or disorder associated with associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, a therapeutically effective amount is an amount effective for treating a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, a therapeutically effective amount is an amount effective for binding SNCA mRNA in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for effecting degradation of SNCA mRNA (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for decreasing an amount of hexokinase 2 (HK2) (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for decreasing an amount of α-synuclein (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

A “prophylactically effective amount” of a compound is an amount sufficient to prevent a condition, or one or more signs and/or symptoms associated with the condition or prevent its recurrence. In certain embodiments, the prophylactically effective amount is an amount that improves overall prophylaxis and/or enhances the prophylactic efficacy of another prophylactic agent. In certain embodiments, a prophylactically effective amount is an amount effective for preventing a disease or disorder associated with an RNA target in a subject in need thereof. In certain embodiments, a prophylactically effective amount is an amount effective for preventing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, a prophylactically effective amount is an amount effective for reducing the risk of developing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, a prophylactically effective amount is an amount effective for binding SNCA mRNA in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for effecting degradation of SNCA mRNA (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for decreasing an amount of hexokinase 2 (HK2) (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for decreasing an amount of α-synuclein (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

The term “gene” refers to a nucleic acid fragment that expresses a protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

“RNA transcript” refers to the product resulting from RNA polymerase-catalyzed transcription of a DNA sequence. When the RNA transcript is a complementary copy of the DNA sequence, it is referred to as the primary transcript, or it may be an RNA sequence derived from post-transcriptional processing of the primary transcript and is referred to as the mature RNA. “Messenger RNA (mRNA)” refers to the RNA that is without introns and can be translated into polypeptides by the cell. “CRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs, inclusive) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “microRNAs” or “miRNA” refers to small non-coding RNAs that are transcribed as primary transcripts that are processed first in the nucleus by a first nuclease to liberate the precursor miRNA, and then in the cytoplasm by a second nuclease to produce the mature miRNA. In certain embodiments, the term “microRNAs” or “miRNA” refers to small non-coding RNAs that are transcribed as primary transcripts that are processed first in the nucleus by Drosha to liberate the precursor miRNA, and then in the cytoplasm by Dicer to produce the mature miRNA.

The term “linker” refers to a bond or a divalent chemical moiety that is bonded to (i.e., that connects) two separate monovalent chemical moieties (e.g., B and R in Formula (I)).

The term “RNA binder” refers to a compound or chemical moiety that is capable of binding to RNA (e.g., an RNA target). In certain embodiments, binding interactions between the RNA binder and RNA are based on structure. In certain embodiments, binding interactions between the RNA binder and RNA are based on structure and not the RNA sequence. In certain embodiments, the RNA binder and RNA form a ternary complex. In certain embodiments, the RNA binder is identified using Inforna or Inforna 2.0, as described in S. P. Velagapudi et al., Nat. Chem. Biol. 2014, 10(4): 291-97 and M. D. Disney et al., ACS Chem. Biol. 2016, 11(6): 1720-28, the contents of which are incorporated herein by reference. In certain embodiments, the RNA binder is identified using two-dimensional combinatorial screening (2DCS), as described in M. D. Disney et al., J. Am. Chem. Soc. 2008, 130(33): 11185-94 and International Patent Application No. PCT/US2018/000020, the contents of which are incorporated herein by reference. In certain embodiments, the RNA binder is identified using a DNA-encoded library (DEL), as described in R. I. Benhamou, et al., Proc. Natl. Acad. Sci. U.S.A. 2022, 119(6) e2114971119, the contents of which are incorporated herein by reference. In certain embodiments, the RNA binder is identified using chemical cross-linking and isolation by pull-down (Chem-CLIP), as described in International Patent Application No. PCT/US2020/070189 and B. M. Suresh, et al., Proc. Natl. Acad. Sci. U.S.A. 2020, 117(52): 33197-203, the contents of which are incorporated herein by reference.

The term “RNase L recruiter” refers to refers to a compound or chemical moiety that is capable of assembling RNase L in the proximity of the RNase L recruiter. In certain embodiments, the RNase L recruiter is capable of assembling active RNase L in the proximity of the RNse L recruiter.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, the present disclosure provides a compound of Formula (I):

B-L-R (I),

or a pharmaceutically acceptable salt, solvate, hydrate, polymorph, co-crystal, tautomer, stereoisomer, isotopically labeled compound, or prodrug thereof, wherein:
B is an RNA binder of Formula (II):

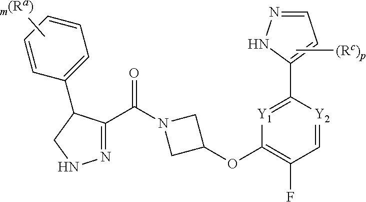

each instance of R^{1A}, R^{1B}, and R^{1C} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NRA)R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.
each of m1, m2, and m3 is independently 0, 1, 2, 3, or 4;
L is a linker; and
R is an RNAse L recruiter.

In certain embodiments, B is an RNA binder of Formula (II):

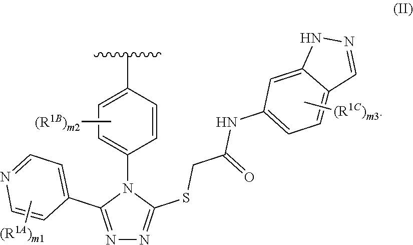

In certain embodiments, B is an RNA binder of Formula (II-a):

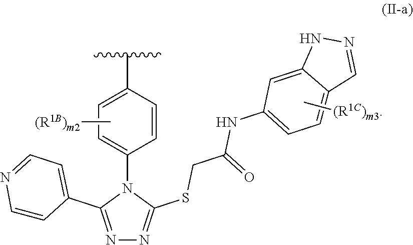

In certain embodiments, B is an RNA binder of Formula (II-b):

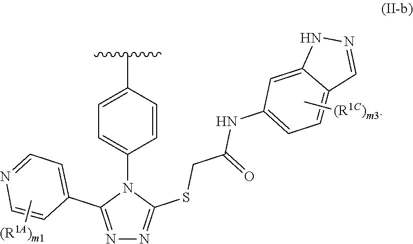

In certain embodiments, B is an RNA binder of Formula (II-c):

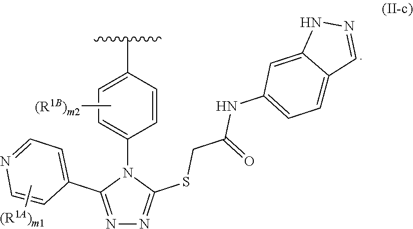

In certain embodiments, B is an RNA binder of Formula (II-d):

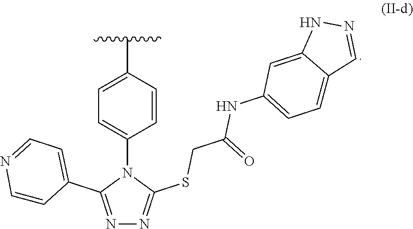 [SMILES: *c1ccc(-n2c(SCC(=O)Nc3ccc4cn[nH]c4c3)nnc2-c2ccncc2)cc1]

In certain embodiments, each occurrence of R^{1A}, R^{1B}, and R^{1C} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, at least one occurrence of RIA is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, at least one occurrence of R^{1B} is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, at least one occurrence of R^{1C} is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, each of m1, m2, and m3 is independently 0, 1, 2, 3, or 4. In certain embodiments, m1 is 0, 1, 2, 3, or 4. In certain embodiments, m1 is 0. In certain embodiments, m1 is 1. In certain embodiments, m1 is 2. In certain embodiments, m1 is 3. In certain embodiments, m1 is 4. In certain embodiments, m2 is 0, 1, 2, 3, or 4. In certain embodiments, m2 is 0. In certain embodiments, m2 is 1. In certain embodiments, m2 is 2. In certain embodiments, m2 is 3. In certain embodiments, m2 is 4. In certain embodiments, m3 is 0, 1, 2, 3, or 4. In certain embodiments, m3 is 0. In certain embodiments, m3 is 1. In certain embodiments, m3 is 2. In certain embodiments, m3 is 3. In certain embodiments, m3 is 4.

In certain embodiments, m1 is 0 and m2 is 0. In certain embodiments, m1 is 1 and m2 is 0. In certain embodiments, m1 is 2 and m2 is 0. In certain embodiments, m1 is 3 and m2 is 0. In certain embodiments, m1 is 4 and m2 is 0. In certain embodiments, m1 is 0 and m2 is 1. In certain embodiments, m1 is 1 and m2 is 1. In certain embodiments, m1 is 2 and m2 is 1. In certain embodiments, m1 is 3 and m2 is 1. In certain embodiments, m1 is 4 and m2 is 1. In certain embodiments, m1 is 0 and m2 is 2. In certain embodiments, m1 is 1 and m2 is 2. In certain embodiments, m1 is 2 and m2 is 2. In certain embodiments, m1 is 3 and m2 is 2. In certain embodiments, m1 is 4 and m2 is 2. In certain embodiments, m1 is 0 and m2 is 3. In certain embodiments, m1 is 1 and m2 is 3. In certain embodiments, m1 is 2 and m2 is 3. In certain embodiments, m1 is 3 and m2 is 3. In certain embodiments, m1 is 4 and m2 is 3. In certain embodiments, m1 is 0 and m2 is 4. In certain embodiments, m1 is 1 and m2 is 4. In certain embodiments, m1 is 2 and m2 is 4. In certain embodiments, m1 is 3 and m2 is 4. In certain embodiments, m1 is 4 and m2 is 4. In certain embodiments, m1 is 0 and m3 is 0. In certain embodiments, m1 is 1 and m3 is 0. In certain embodiments, m1 is 2 and m3 is 0. In certain embodiments, m1 is 3 and m3 is 0. In certain embodiments, m1 is 4 and m3 is 0. In certain embodiments, m1 is 0 and m3 is 1. In certain embodiments, m1 is 1 and m3 is 1. In certain embodiments, m1 is 2 and m3 is 1. In certain embodiments, m1 is 3 and m3 is 1. In certain embodiments, m1 is 4 and m3 is 1. In certain embodiments, m1 is 0 and m3 is 2. In certain embodiments, m1 is 1 and m3 is 2. In certain embodiments, m1 is 2 and m3 is 2. In certain embodiments, m1 is 3 and m3 is 2. In certain embodiments, m1 is 4 and m3 is 2. In certain embodiments, m1 is 0 and m3 is 3. In certain embodiments, m1 is 1 and m3 is 3. In certain embodiments, m1 is 2 and m3 is 3. In certain embodiments, m1 is 3 and m3 is 3. In certain embodiments, m1 is 4 and m3 is 3. In certain embodiments, m1 is 0 and m3 is 4. In certain embodiments, m1 is 1 and m3 is 4. In certain embodiments, m1 is 2 and m3 is 4. In certain embodiments, m1 is 3 and m3 is 4. In certain embodiments, m1 is 4 and m3 is 4. In certain embodiments, m2 is 0 and m3 is 0. In certain embodiments, m2 is 1 and m3 is 0. In certain embodiments, m2 is 2 and m3 is 0. In certain embodiments, m2 is 3 and m3 is 0. In certain embodiments, m2 is 4 and m3 is 0. In certain embodiments, m2 is 0 and m3 is 1. In certain embodiments, m2 is 1 and m3 is 1. In certain embodiments, m2 is 2 and m3 is 1. In certain embodiments, m2 is 3 and m3 is 1. In certain embodiments, m2 is 4 and m3 is 1. In certain embodiments, m2 is 0 and m3 is 2. In certain embodiments, m2 is 1 and m3 is 2. In certain embodiments, m2 is 2 and m3 is 2. In certain embodiments, m2 is 3 and m3 is 2. In certain embodiments, m2 is 4 and m3 is 2. In certain embodiments, m2 is 0 and m3 is 3. In certain embodiments, m2 is 1 and m3 is 3. In certain embodiments, m2 is 2 and m3 is 3. In certain embodiments, m2 is 3 and m3 is 3. In certain embodiments, m2 is 4 and m3 is 3. In certain embodiments, m2 is 0 and m3 is 4. In certain embodiments, m2 is 1 and m3 is 4. In certain embodiments, m2 is 2 and m3 is 4. In certain embodiments, m2 is 3 and m3 is 4. In certain embodiments, m2 is 4 and m3 is 4. In certain embodiments, m1 is 0, m2 is 0, and m3 is 0.

In certain embodiments, L is a linker. In certain embodiments, L is a bond, optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof. In certain embodiments, L is a bond. In certain embodiments, L is optionally substituted alkylene. In certain embodiments, L is optionally substituted alkenylene. In certain embodiments, L is optionally substituted alkynylene. In certain embodiments, L is optionally substituted heteroalkylene. In certain embodiments, L is optionally substituted heteroalkenylene. In certain embodiments, L is optionally substituted heteroalkynylene. In certain embodiments, L is optionally substituted heterocyclylene. In certain embodiments, L is optionally substituted carbocyclylene. In certain embodiments, L is optionally substituted arylene. In certain embodiments, L is optionally substituted heteroarylene. In certain embodiments, L is optionally substituted alkylene, optionally substituted heteroalkylene, or a combination thereof.

In certain embodiments, L comprises a moiety of formula

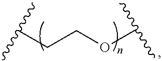

wherein n is 0, 1, 2, 3, 4, 5, 6, 7, 8, 9, or 10. In certain embodiments, L comprises a moiety of formula

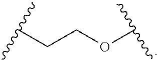 [SMILES: *CCO*]

In certain embodiments, L comprises a moiety of formula

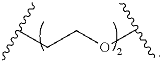

In certain embodiments, L comprises a moiety of formula

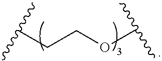

In certain embodiments, L comprises a moiety of formula

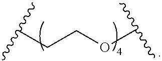

In certain embodiments, L comprises a moiety of formula

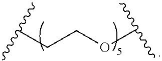

In certain embodiments, L comprises a moiety of formula

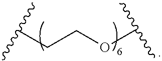

In certain embodiments, L comprises a moiety of formula

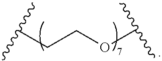

In certain embodiments, L comprises a moiety of formula

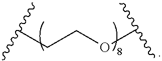

In certain embodiments, L comprises a moiety of formula

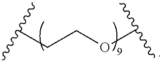

In certain embodiments, L comprises a moiety of formula

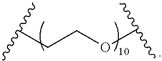

In certain embodiments, B is an RNA binder of Formula (II-d), and L comprises a moiety of formula

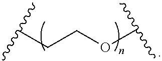

In certain embodiments, B is an RNA binder of Formula (II-d), and L comprises a moiety of formula

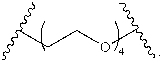

In certain embodiments, L comprises a moiety of formula

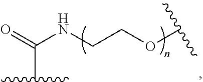

wherein n is 0, 1, 2, 3, 4, 5, 6, 7, 8, 9, or 10. In certain embodiments, L comprises a moiety of formula

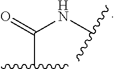 [SMILES: *NC(*)=O]

In certain embodiments, L comprises a moiety of formula

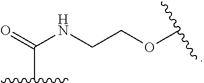 [SMILES: *OCCNC(*)=O]

In certain embodiments, L comprises a moiety of formula

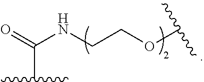

In certain embodiments, L comprises a moiety of formula

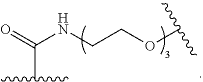

In certain embodiments, L comprises a moiety of formula

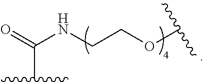

In certain embodiments, L comprises a moiety of formula

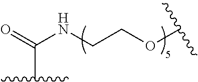

In certain embodiments, L comprises a moiety of formula

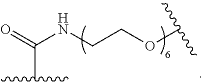

In certain embodiments, L comprises a moiety of formula

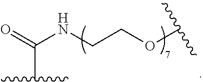

In certain embodiments, L comprises a moiety of formula

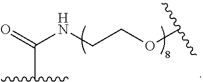

In certain embodiments, L comprises a moiety of formula

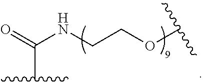

In certain embodiments, L comprises a moiety of formula

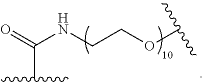

In certain embodiments, B is an RNA binder of Formula (II-d), and L comprises a moiety of formula

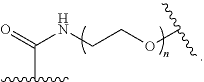

In certain embodiments, B is an RNA binder of Formula (II-d), and L comprises a moiety of formula

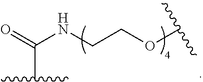

In certain embodiments, L is of Formula (III):

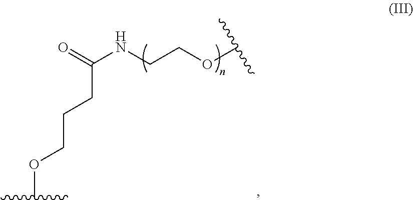

wherein n is 0, 1, 2, 3, 4, 5, 6, 7, 8, 9, or 10. In certain embodiments, n is 0, 1, 2, 3, 4, 5, 6, 7, 8, 9, or 10. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, L is of formula

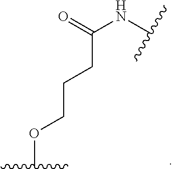 [SMILES: *NC(=O)CCCO*]

In certain embodiments, L is of formula

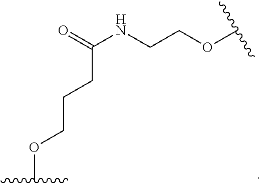 [SMILES: *OCCCC(=O)NCCO*]

In certain embodiments, L is of formula.

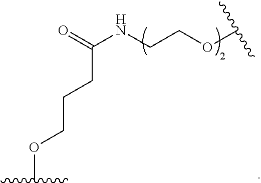

In certain embodiments, L is of formula

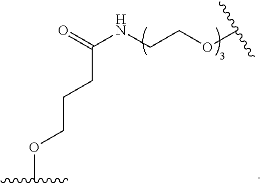

In certain embodiments, L is of formula

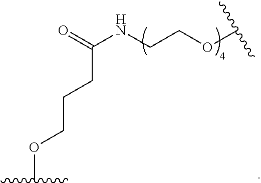

In certain embodiments, L is of formula

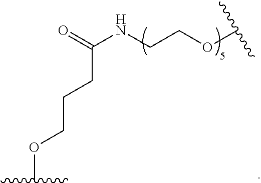

In certain embodiments, L is of formula

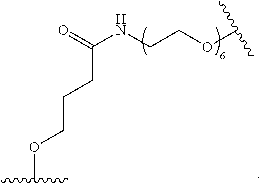

In certain embodiments, L is of formula

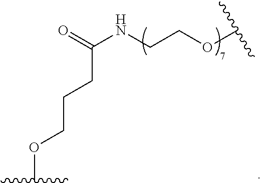

In certain embodiments, L is of formula

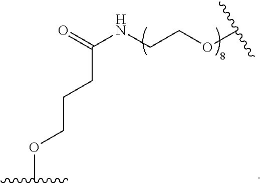

In certain embodiments, L is of formula

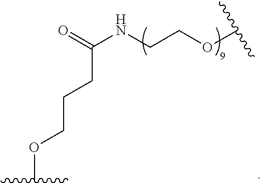

In certain embodiments, L is of formula

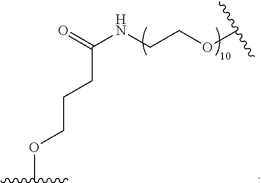

In certain embodiments, B is an RNA binder of Formula (II-d), and L is of formula

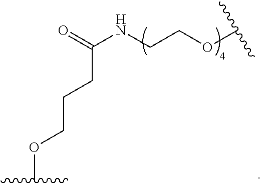

In certain embodiments, R is an RNAse L recruiter. In certain embodiments, R is an RNAse L recruiter of Formulae (IV-a) or (IV-b):

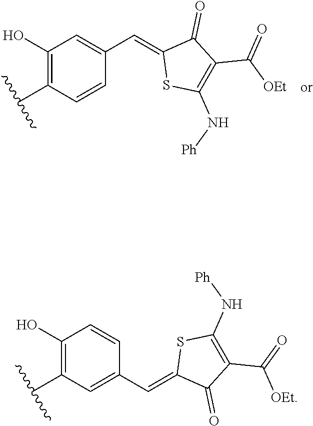 [SMILES: *c1ccc(/C=C2\SC(Nc3ccccc3)=C(C(=O)OCC)C2=O)cc1O ; *c1cc(/C=C2\SC(Nc3ccccc3)=C(C(=O)OCC)C2=O)ccc1O]

In certain embodiments, R is an RNAse L recruiter of Formula (IV-a):

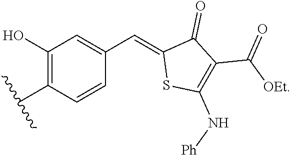 [SMILES: *c1ccc(/C=C2\SC(Nc3ccccc3)=C(C(=O)OCC)C2=O)cc1O]

In certain embodiments, R is an RNAse L recruiter of Formula (IV-b):

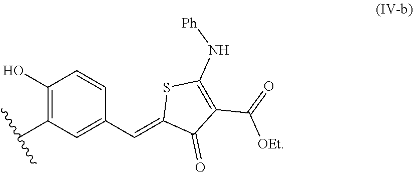 [SMILES: *c1cc(/C=C2\SC(Nc3ccccc3)=C(C(=O)OCC)C2=O)ccc1O]

In certain embodiments, R is an RNAse L recruiter of Formula (IV-a), and B is an RNA binder of Formula (II-d). In certain embodiments, R is an RNAse L recruiter of Formula (IV-a), and L comprises a moiety of formula

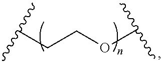

In certain embodiments, R is an RNAse L recruiter of Formula (IV-a), and L comprises a moiety of formula

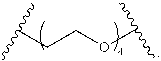

In certain embodiments, R is an RNAse L recruiter of Formula (IV-a), and L comprises a moiety of formula

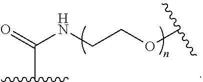

In certain embodiments, R is an RNAse L recruiter of Formula (IV-a), and L comprises a moiety of formula

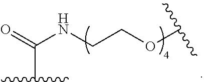

In certain embodiments, R is an RNAse L recruiter of Formula (IV-a), and L is of formula

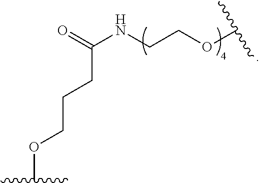

In certain embodiments, R is an RNAse L recruiter of Formula (IV-a), B is an RNA binder of Formula (II-d), and L comprises a moiety of formula

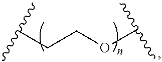

In certain embodiments, R is an RNAse L recruiter of Formula (IV-a), B is an RNA binder of Formula (II-d), and L comprises a moiety of formula

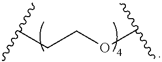

In certain embodiments, R is an RNAse L recruiter of Formula (IV-a), B is an RNA binder of Formula (II-d), and L comprises a moiety of formula

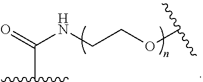

In certain embodiments, R is an RNAse L recruiter of Formula (IV-a), B is an RNA binder of Formula (II-d), and L comprises a moiety of formula

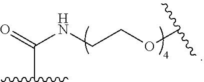

In certain embodiments, R is an RNAse L recruiter of Formula (IV-a), B is an RNA binder of Formula (II-d), and L is of formula

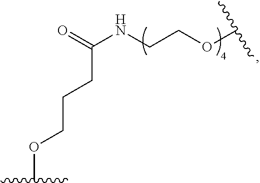

In certain embodiments, each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is hydrogen. In certain embodiments, at least one occurrence of R^{A} is optionally substituted acyl. In certain embodiments, at least one occurrence of R^{A} is optionally substituted C₁₋₁₂ alkyl. In certain embodiments, at least one occurrence of R^{A} is optionally substituted C₁₋₆ alkyl. In certain embodiments, at least one occurrence of R^{A} is unsubstituted C₁₋₆ alkyl. In certain embodiments, at least one occurrence of R^{A} is substituted C₁₋₆ alkyl. In certain embodiments, at least one occurrence of R^{A} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least one occurrence of R^{A} is optionally substituted C₂₋₁₂ alkenyl. In certain embodiments, at least one occurrence of R^{A} is optionally substituted C₂₋₆ alkenyl. In certain embodiments, at least one occurrence of R^{A} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R^{A} is optionally substituted C₂₋₁₂ alkynyl. In certain embodiments, at least one occurrence of R^{A} is optionally substituted C₂₋₆ alkynyl. In certain embodiments, at least one occurrence of R^{A} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R^{A} is optionally substituted heteroC₁₋₁₂ alkyl. In certain embodiments, at least one occurrence of R^{A} is optionally substituted heteroC₁₋₆ alkyl. In certain embodiments, at least one occurrence of R^{A} is optionally substituted heteroC₁₋₁₂ alkenyl. In certain embodiments, at least one occurrence of R^{A} is optionally substituted heteroC₁₋₆ alkenyl. In certain embodiments, at least one occurrence of R^{A} is optionally substituted heteroC₁₋₁₂ alkynyl. In certain embodiments, at least one occurrence of R^{A} is optionally substituted heteroC₁₋₆ alkynyl. In certain embodiments, at least one occurrence of R^{A} is optionally substituted C₃₋₁₄ cycloalkyl. In certain embodiments, at least one occurrence of R^{A} is optionally substituted 5-10 membered heterocyclyl. In certain embodiments, at least one occurrence of R^{A} is optionally substituted 6-14 membered aryl. In certain embodiments, at least one occurrence of R^{A} is optionally substituted 5-14 membered heteroaryl. In certain embodiments, at least one occurrence of R^{A} is a nitrogen protecting group when attached to a nitrogen atom. In certain embodiments, at least one occurrence of R^{A} is an oxygen protecting group when attached to an oxygen atom. In certain embodiments, at least one occurrence of R^{A} is a sulfur protecting group when attached to a sulfur atom. In certain embodiments, at least two occurrences of R^{A} are joined together with their intervening atom to form an optionally substituted 5-10 membered heterocyclic ring. In certain embodiments, at least two occurrences of R^{A} are joined together with their intervening atom to form an optionally substituted 5-14 membered heteroaryl ring.

In certain embodiments, the compound of Formula (I) is of Formula (I-a):

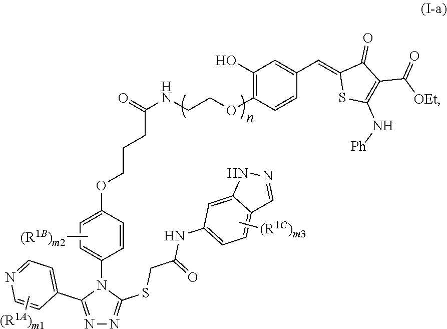

or a pharmaceutically acceptable salt, solvate, hydrate, polymorph, co-crystal, tautomer, stereoisomer, isotopically labeled compound, or prodrug thereof, wherein:
each instance of R^{1A}, R^{1B}, and R^{1C} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.
each of m1, m2, and m3 is independently 0, 1, 2, 3, or 4; and
n is 0, 1, 2, 3, 4, 5, 6, 7, 8, 9, or 10.

In certain embodiments, the compound of Formula (I-a) is of Formula (I-a-i):

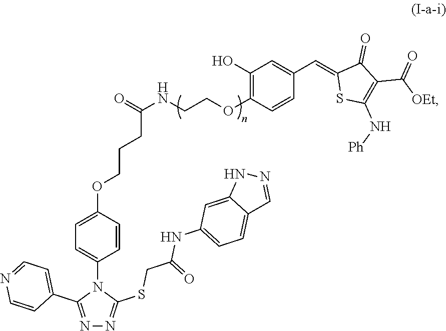

or a pharmaceutically acceptable salt, solvate, hydrate, polymorph, co-crystal, tautomer, stereoisomer, isotopically labeled compound, or prodrug thereof.

In certain embodiments, the compound of Formula (I) is of formula:

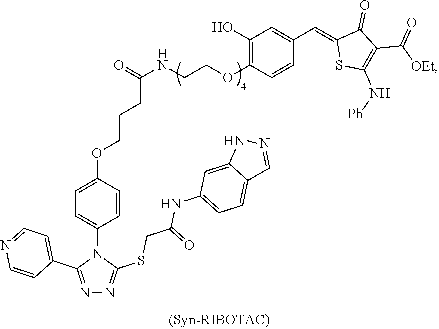

or a pharmaceutically acceptable salt, solvate, hydrate, polymorph, co-crystal, tautomer, stereoisomer, isotopically labeled compound, or prodrug thereof.

In certain embodiments, the compound of Formula (I) is of Formula (I-b):

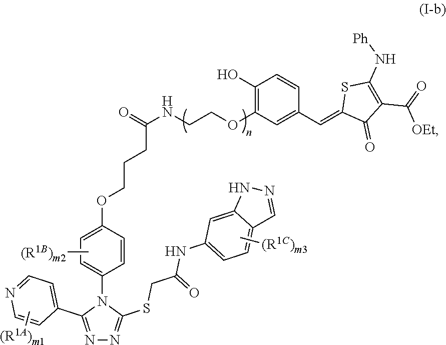

or a pharmaceutically acceptable salt, solvate, hydrate, polymorph, co-crystal, tautomer, stereoisomer, isotopically labeled compound, or prodrug thereof, wherein:
each instance of R^{1A}, R^{1B}, and R^{1C} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.
each of m1, m2, and m3 is independently 0, 1, 2, 3, or 4; and
n is 0, 1, 2, 3, 4, 5, 6, 7, 8, 9, or 10.

In certain embodiments, the compound of Formula (I-b) is of Formula (I-b-i):

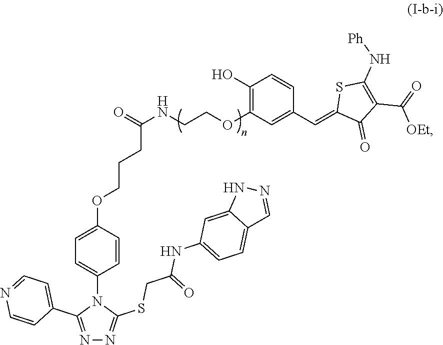

or a pharmaceutically acceptable salt, solvate, hydrate, polymorph, co-crystal, tautomer, stereoisomer, isotopically labeled compound, or prodrug thereof.

In certain embodiments, the compound of Formula (I) is of formula:

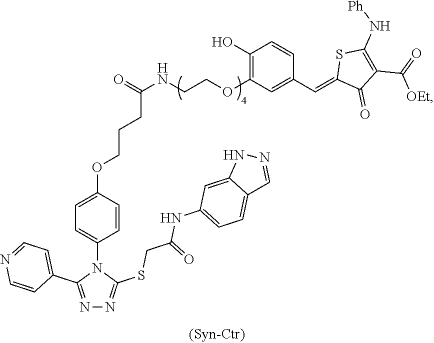

or a pharmaceutically acceptable salt, solvate, hydrate, polymorph, co-crystal, tautomer, stereoisomer, isotopically labeled compound, or prodrug thereof.

In another aspect, the present disclosure provides compounds of Formula:

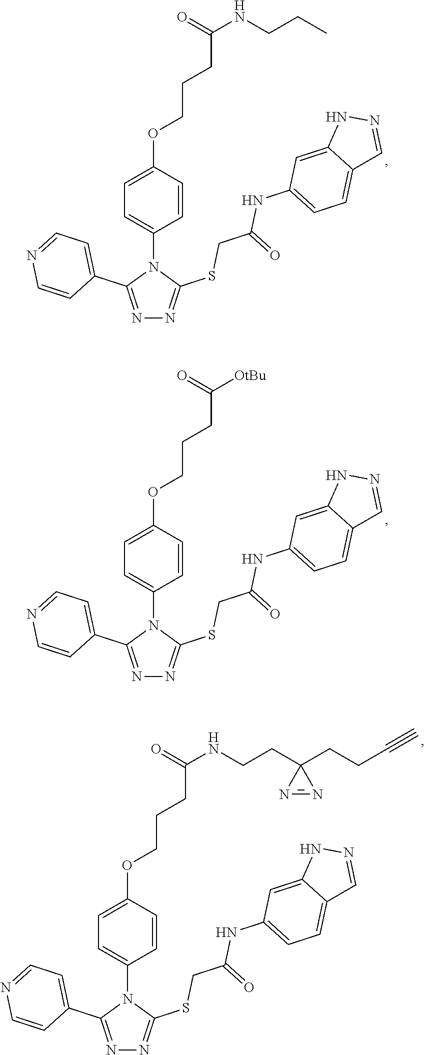 [SMILES: CCCNC(=O)CCCOc1ccc(-n2c(SCC(=O)Nc3ccc4cn[nH]c4c3)nnc2-c2ccncc2)cc1 ; CC(C)(C)OC(=O)CCCOc1ccc(-n2c(SCC(=O)Nc3ccc4cn[nH]c4c3)nnc2-c2ccncc2)cc1 ; C#CCCC1(CCNC(=O)CCCOc2ccc(-n3c(SCC(=O)Nc4ccc5cn[nH]c5c4)nnc3-c3ccncc3)cc2)N=N1]

or a pharmaceutically acceptable salt, solvate, hydrate, polymorph, co-crystal, tautomer, stereoisomer, isotopically labeled compound, or prodrug thereof.

In certain embodiments, a provided compound (a compound described herein, a compound of the present disclosure) is a compound of any of the formulae herein (e.g., Formula (I)) or pharmaceutically acceptable salt, solvate, hydrate, polymorph, co-crystal, tautomer, stereoisomer, isotopically labeled compound, or prodrug thereof. In certain embodiments, a provided compound is a compound of any of the formulae herein (e.g., Formula (I)), or a pharmaceutically acceptable salt or tautomer thereof. In certain embodiments, a provided compound is a compound of any of the formulae herein (e.g., Formula (I)), or a pharmaceutically acceptable salt thereof. In certain embodiments, a provided compound is a compound of any of the formulae herein (e.g., Formula (I)), or a salt thereof.

### Pharmaceutical Compositions and Kits

In one aspect, the present disclosure provides pharmaceutical compositions comprising a provided compound. In some embodiments, the pharmaceutical composition comprises one or more excipients. In certain embodiments, the pharmaceutical compositions described herein comprise a provided compound and an excipient.

In certain embodiments, the pharmaceutical composition comprises an effective amount of the provided compound. In certain embodiments, the effective amount is a therapeutically effective amount. In certain embodiments, the effective amount is a prophylactically effective amount.

In certain embodiments, the effective amount is an amount effective for binding an RNA target in a subject in need thereof or in a cell, tissue, or biological sample. In certain embodiments, the effective amount is an amount effective for effecting degradation of an RNA target in a subject in need thereof or in a cell, tissue, or biological sample. In certain embodiments, the RNA target is SNCA mRNA. In certain embodiments, the effective amount is an amount effective for treating a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, the effective amount is an amount effective for preventing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, the effective amount is an amount effective for reducing the risk of developing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof.

In certain embodiments, the subject is an animal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years, inclusive. In certain embodiments, the subject is a human younger than 2 years. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for decreasing an amount of SNCA mRNA (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the effective amount is an amount effective for decreasing an amount of SNCA mRNA by a range between a percentage described in this paragraph and another percentage described in this paragraph, inclusive.

In certain embodiments, the effective amount is an amount effective for decreasing an amount of hexokinase 2 (HK2) (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the effective amount is an amount effective for decreasing an amount of hexokinase 2 (HK2) by a range between a percentage described in this paragraph and another percentage described in this paragraph, inclusive.

In certain embodiments, the effective amount is an amount effective for decreasing an amount of α-synuclein (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the effective amount is an amount effective for decreasing an amount of α-synuclein by a range between a percentage described in this paragraph and another percentage described in this paragraph, inclusive.

In certain embodiments, the pharmaceutical composition is for use in treating a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, the pharmaceutical composition is for use in preventing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, the pharmaceutical composition is for use in binding an RNA target in a subject in need thereof or in a cell, tissue, or biological sample. In certain embodiments, the pharmaceutical composition is for use in effecting degradation of an RNA target in a subject in need thereof or in a cell, tissue, or biological sample.

A provided compound or pharmaceutical composition, as described herein, can be administered in combination with one or more additional pharmaceutical agents (e.g., therapeutically and/or prophylactically active agents). The provided compounds or pharmaceutical compositions can be administered in combination with additional pharmaceutical agents that improve their activity (e.g., activity (e.g., potency and/or efficacy) in treating a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof, in preventing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof, and/or in reducing the risk of developing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof), improve bioavailability, improve safety, reduce drug resistance, reduce and/or modify metabolism, inhibit excretion, and/or modify distribution in a subject or cell. It will also be appreciated that the additional pharmaceutical agents employed may achieve a desired effect for the same disorder, and/or it may achieve different effects. In certain embodiments, a pharmaceutical composition described herein including a provided compound described herein and an additional pharmaceutical agent exhibit a synergistic effect that is absent in a pharmaceutical composition including one of the provided compounds and the additional pharmaceutical agent, but not both. In some embodiments, the additional pharmaceutical agent achieves a desired effect for the same disorder. In some embodiments, the additional pharmaceutical agent achieves different effects.

The provided compound or pharmaceutical composition can be administered concurrently with, prior to, or subsequent to one or more additional pharmaceutical agents, which are different from the compound or pharmaceutical composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides, synthetic proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or disorder associated with SNCA mRNA (e.g., a neurodegenerative disease (e.g., Parkinson's Disease, Alzheimer's disease, Lewy bodies' disease, Muscular System Atrophy)).

Each additional pharmaceutical agent may be administered at a dose and/or on a time schedule determined for that pharmaceutical agent. The additional pharmaceutical agents may also be administered together with each other and/or with the compound or pharmaceutical composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

In certain embodiments, the provided compound or pharmaceutical composition is a solid. In certain embodiments, the provided compound or pharmaceutical composition is a powder. In certain embodiments, the provided compound or pharmaceutical composition can be dissolved in a liquid to make a solution. In certain embodiments, the provided compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

Pharmaceutical compositions described herein can be prepared by any method known in the art of pharmacology. In general, such preparatory methods include the steps of bringing the composition comprising a provided compound (i.e., the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the provided compound, pharmaceutically acceptable excipient, agent, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) agent, inclusive.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid pharmaceutical compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Pharmaceutical compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Suitable devices for use in delivering injectable pharmaceutical compositions described herein include short needle devices. Injectable pharmaceutical compositions can be administered by devices which limit the effective penetration length of a needle into the skin. Alternatively or additionally, conventional syringes can be used in the classical mantoux method of administration. Jet injection devices which deliver liquid formulations via a liquid jet injector and/or via a needle. Ballistic powder/particle delivery devices which use compressed gas to accelerate the compound in powder form are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such pharmaceutical compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder pharmaceutical compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such pharmaceutical compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Provided compounds are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The provided compounds and pharmaceutical compositions provided herein can be administered by any route, including enteral (e.g., oral), parenteral, intravenous, intramuscular, intraarticular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are intraarticular administration, oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a provided compound required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, a pharmaceutical composition comprising a provided compound is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the pharmaceutical composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Dose ranges as described herein provide guidance for the administration of provided compounds or pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Also encompassed by the present disclosure are kits (e.g., pharmaceutical packs). In certain embodiments, the kit comprises a provided compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the provided compound or pharmaceutical composition included in the first container and the excipient or additional pharmaceutical agent included in the second container are combined to form one unit dosage form. In some embodiments, the provided compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the provided compound or pharmaceutical composition to a subject (e.g., a subject in need of treatment or prevention of a disease described herein). In certain embodiments, the instructions are for contacting a biological sample or cell with the provided compound or pharmaceutical composition. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

In certain embodiments, the kits and instructions provide for treating a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, the kits and instructions provide for preventing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, the kits and instructions provide for reducing the risk of developing a disease or disorder associated with SNCA mRNA (e.g., a neurodegenerative disease (e.g., Parkinson's Disease, Alzheimer's disease, Lewy bodies' disease, Muscular System Atrophy)) in a subject in need thereof. In certain embodiments, the kits and instructions provide for binding SNCA mRNA in a subject in need thereof or in a cell, tissue, or biological sample. In certain embodiments, the kits and instructions provide for effecting degradation of SNCA mRNA in a subject in need thereof or in a cell, tissue, or biological sample.

A kit described herein may include one or more additional pharmaceutical agents described herein as a separate pharmaceutical composition.

Another object of the present disclosure is the use of a compound as described herein in the manufacture of a medicament for use in the treatment of a disorder or disease described herein. Another object of the present disclosure is the use of a compound as described herein for use in the treatment of a disorder or disease described herein.

### Methods of Treatment and Prevention

In another aspect, the present disclosure provides methods of treating or preventing a disease in a subject in need thereof, comprising administering to the subject in need thereof a provided compound or pharmaceutical composition. In certain embodiments, the present disclosure provides methods of treating a disease in a subject in need thereof, comprising administering to the subject in need thereof a provided compound or pharmaceutical composition. In certain embodiments, the present disclosure provides methods of preventing a disease in a subject in need thereof, comprising administering to the subject in need thereof a provided compound or pharmaceutical composition. In certain embodiments, the disease is associated with SNCA mRNA (e.g., a neurodegenerative disease (e.g., Parkinson's Disease, Alzheimer's disease, Lewy bodies' disease, Muscular System Atrophy)).

In another aspect, the present disclosure provides a provided compound or pharmaceutical composition for use in treating or preventing a disease in a subject in need thereof. In another aspect, the present disclosure provides a provided compound or pharmaceutical composition for use in treating a disease in a subject in need thereof. In another aspect, the present disclosure provides a provided compound or pharmaceutical composition for use in preventing a disease in a subject in need thereof. In certain embodiments, the disease is associated with SNCA mRNA (e.g., a neurodegenerative disease (e.g., Parkinson's Disease, Alzheimer's disease, Lewy bodies' disease, Muscular System Atrophy)).

In another aspect, the present disclosure provides a provided compound or pharmaceutical composition for use in the manufacture of a medicament for treatment or prevention of a disease in a subject in need thereof. In another aspect, the present disclosure provides a provided compound or pharmaceutical composition for use in the manufacture of a medicament for treatment of a disease in a subject in need thereof. In another aspect, the present disclosure provides a provided compound or pharmaceutical composition for use in the manufacture of a medicament for prevention of a disease in a subject in need thereof. In certain embodiments, the disease is associated with SNCA mRNA (e.g., a neurodegenerative disease (e.g., Parkinson's Disease, Alzheimer's disease, Lewy bodies' disease, Muscular System Atrophy)).

In certain embodiments, the disease is associated with SNCA mRNA (e.g., a neurodegenerative disease (e.g., Parkinson's Disease, Alzheimer's disease, Lewy bodies' disease, Muscular System Atrophy)). In certain embodiments, the disease is a neurodegenerative disease (e.g., Parkinson's Disease, Alzheimer's disease, Lewy bodies' disease, Muscular System Atrophy). In certain embodiments, the disease is Parkinson's Disease. In certain embodiments, the neurodegenerative disease is Parkinson's Disease. In certain embodiments, the neurodegenerative disease is Alzheimer's disease. In certain embodiments, the neurodegenerative disease is Lewy bodies' disease. In certain embodiments, the neurodegenerative disease is Muscular System Atrophy.

### Methods of Binding RNase L, Binding an RNA Target, and Effecting Degradation of an RNA Target

In another aspect, the present disclosure provides methods of binding RNase L in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of binding RNase L in a subject in need thereof, comprising administering to the subject in need thereof an effective amount of a provided compound or composition. In certain embodiments, the present disclosure provides methods of binding RNase L in a cell, tissue, or biological sample, comprising contacting the cell, tissue, or biological sample with an effective amount of a provided compound or composition.

In certain embodiments, binding RNase L comprises activating RNase L (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%). In certain embodiments, binding RNase L comprises inducing RNase L dimerization (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%).

In another aspect, the present disclosure provides methods of binding an RNA target in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of binding an RNA target in a subject in need thereof, comprising administering to the subject in need thereof an effective amount of a provided compound or composition. In certain embodiments, the present disclosure provides methods of binding an RNA target in a cell, tissue, or biological sample, comprising contacting the cell, tissue, or biological sample with an effective amount of a provided compound or composition. In certain embodiments, the present disclosure provides a provided compound or composition for use in binding an RNA target in a subject in need thereof or in a cell, tissue, or biological sample. In certain embodiments, the present disclosure provides a provided compound or composition for use in the manufacture of a medicament for binding an RNA target in a subject in need thereof or in a cell, tissue, or biological sample.

In another aspect, the present disclosure provides methods of effecting degradation of an RNA target in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of effecting degradation of an RNA target in a subject in need thereof, comprising administering to the subject in need thereof an effective amount of a provided compound or composition. In certain embodiments, the present disclosure provides methods of effecting degradation of an RNA target in a cell, tissue, or biological sample, comprising contacting the cell, tissue, or biological sample with an effective amount of a provided compound or composition. In certain embodiments, the present disclosure provides a provided compound or composition for use in effecting degradation of an RNA target in a subject in need thereof or in a cell, tissue, or biological sample. In certain embodiments, the present disclosure provides a provided compound or composition for use in the manufacture of a medicament for effecting degradation of an RNA target in a subject in need thereof or in a cell, tissue, or biological sample.

In certain embodiments, the method further comprises recruiting RNase L. In certain embodiments, the method further comprises activating RNase L (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%). In certain embodiments, the method further comprises inducing RNase L dimerization (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%).

In certain embodiments, the method comprises cleaving the RNA target (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the method comprises binding the RNA target (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the method comprises decreasing an amount of the RNA target (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the method comprises effecting degradation of the RNA target (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

In certain embodiments, the RNA target is SNCA mRNA. In certain embodiments, the method further comprises decreasing an amount of SNCA mRNA (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the amount of SNCA mRNA is decreased by at least about 48%. In certain embodiments, the method comprises cleaving SNCA mRNA (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

In certain embodiments, the binding or effecting degradation is selective for SNCA mRNA compared to other mRNA. In certain embodiments, the binding is selective for SNCA mRNA compared to other mRNA. In certain embodiments, the effecting degradation is selective for SNCA mRNA compared to other mRNA. In certain embodiments, the binding or effecting degradation is selective for SNCA mRNA compared to APP mRNA, ferritin mRNA, and TfR mRNA. In certain embodiments, the binding is selective for SNCA mRNA compared to APP mRNA, ferritin mRNA, and TfR mRNA. In certain embodiments, the effecting degradation is selective for SNCA mRNA compared to APP mRNA, ferritin mRNA, and TfR mRNA.

In certain embodiments, the provided compound or composition interacts with an SNCA mRNA iron-responsive element (IRE). In certain embodiments, the SNCA mRNA IRE is located in the SNCA mRNA 5′ untranslated region (5′ UTR). In certain embodiments, the SNCA mRNA IRE comprises an A bulge binding pocket.

In certain embodiments, the method further comprises a cytoprotective effect. In certain embodiments, the method further comprises decreasing an amount of hexokinase 2 (HK2) (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the method further comprises decreasing an amount of α-synuclein (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the amount of α-synuclein is decreased by at least about 63%. In certain embodiments, the α-synuclein is decreased in Parkinson's Disease patient-derived dopaminergic neurons. In certain embodiments, the method further comprises rescuing expression of genes abnormally expressed in Parkinson's Disease patient-derived dopaminergic neurons. In certain embodiments, about 50% of genes significantly downregulated in Parkinson's Disease patient-derived dopaminergic neurons are increased. In certain embodiments, about 50% of genes significantly upregulated in Parkinson's Disease patient-derived dopaminergic neurons are decreased. In certain embodiments, about 50% of genes significantly downregulated in Parkinson's Disease patient-derived dopaminergic neurons are increased; and/or about 50% of genes significantly upregulated in Parkinson's Disease patient-derived dopaminergic neurons are decreased.

In certain embodiments, the cell, tissue, or biological sample is in vivo. In certain embodiments, the cell, tissue, or biological sample is in vitro.

### Methods of Preparation

In another aspect, the present disclosure provides methods of preparing a compound of Formulae (I-a) or (I-b):

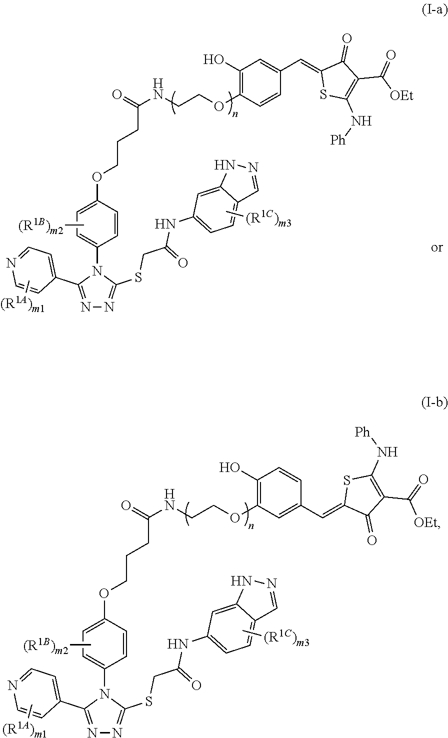

or a pharmaceutically acceptable salt, solvate, hydrate, polymorph, co-crystal, tautomer, stereoisomer, isotopically labeled compound, or prodrug thereof, comprising reacting a compound of Formulae (V-a) or (V-b):

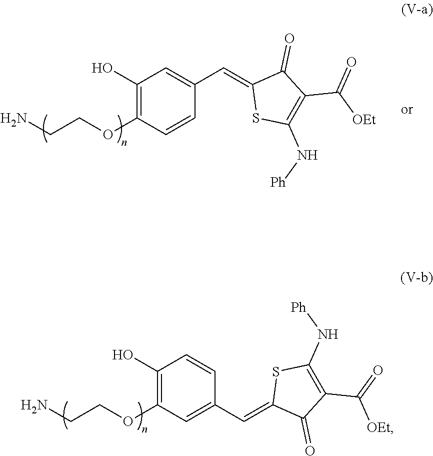

or a salt thereof, with a compound of Formula (VI):

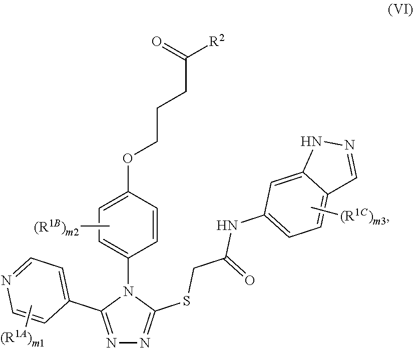

or a salt thereof, wherein:
R² is halogen or —OR^{2a};
R^{2a} is hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each occurrence of R^{1A}, R^{1B}, and R^{1C} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.
each of m1, m2, and m3 is independently 0, 1, 2, 3, or 4; and
n is 0, 1, 2, 3, 4, 5, 6, 7, 8, 9, or 10.

In certain embodiments, R^{2a} is optionally substituted alkyl. In certain embodiments, R^{2a} is optionally substituted C₁₋₆ alkyl. In certain embodiments, R^{2a} is tert-butyl. In certain embodiments, R² is —OtBu. In certain embodiments, m1 is 0. In certain embodiments, m2 is 0. In certain embodiments, m3 is 0. In certain embodiments, each of m1, m2, and m3 is 0. In certain embodiments, n is 4.

In certain embodiments, the compound of Formula (V-a) is of Formula (V-a-i):

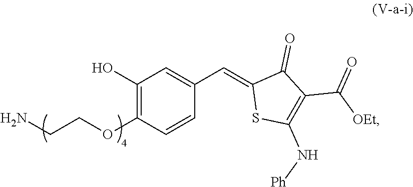

or salt thereof. In certain embodiments, the compound of Formula (V-b) is of Formula (V-b-i):

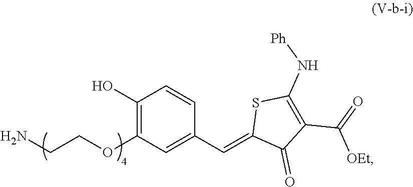

or salt thereof.

In certain embodiments, the compound of Formula (VI) is of Formula (VI-a):

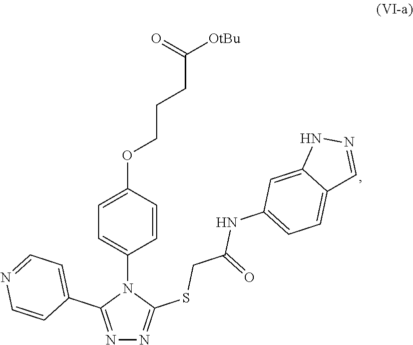 [SMILES: CC(C)(C)OC(=O)CCCOc1ccc(-n2c(SCC(=O)Nc3ccc4cn[nH]c4c3)nnc2-c2ccncc2)cc1]

or a salt thereof.

In certain embodiments, the method further comprises alkylating a compound of Formula (VII):

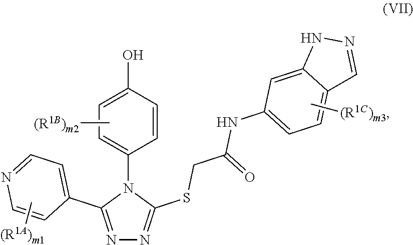

or a salt thereof, to provide the compound of Formula (VI), or salt thereof, wherein:
each instance of R^{1A}, R^{1B}, and R^{1C} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
each of m1, m2, and m3 is independently 0, 1, 2, 3, or 4.

In certain embodiments, the compound of Formula (VII) is of Formula (VII-a):

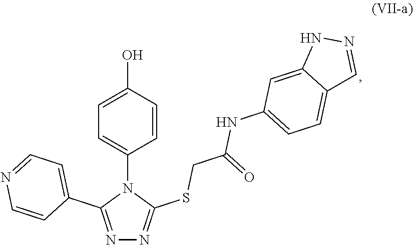 [SMILES: O=C(CSc1nnc(-c2ccncc2)n1-c1ccc(O)cc1)Nc1ccc2cn[nH]c2c1]

or a salt thereof.

In certain embodiments, the method further comprises alkylating a compound of formula:

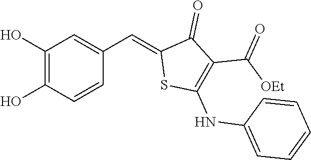 [SMILES: CCOC(=O)C1=C(Nc2ccccc2)S/C(=C\c2ccc(O)c(O)c2)C1=O]

or a salt thereof, to provide the compound of formula:

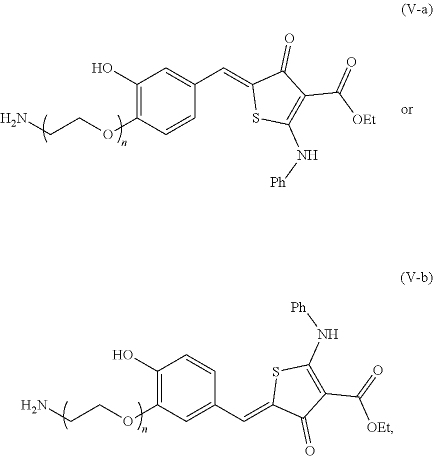

or a salt thereof, wherein n is 0, 1, 2, 3, 4, 5, 6, 7, 8, 9, or 10.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Converting Synucleozid-2.0 into a Ribonuclease Targeting Chimera Enhances Cytoprotectivity

One approach to enhance the potency of RNA binders is to tether them to ribonuclease-recruiting small molecule, affording a ribonuclease recruiting chimera (RiboTAC).⁴⁸ The RNA-binding module drives the interaction with the SNCA IRE while the ribonuclease-recruiting small molecule module dimerizes and activates monomeric RNase L (where L denotes latent) in proximity of the target RNA to induce its cleavage. Previous studies have shown that RiboTACs degrade the target sub-stoichiometrically and are more selective^{48,49} than the RNA-binding small molecule from which they are derived. The observed enhancement in selectivity is a composite of the specificity of the RNA-binding module, the substrate specificity of RNase L, and the juxtaposition of the binding site of the RNA-binding module and a site sensitive to RNase L cleavage.⁴⁹

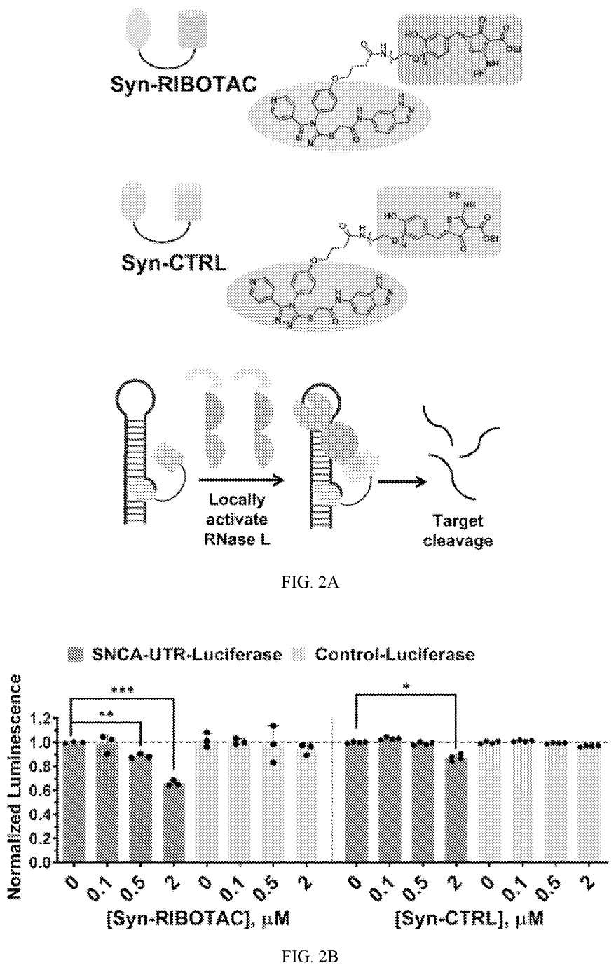

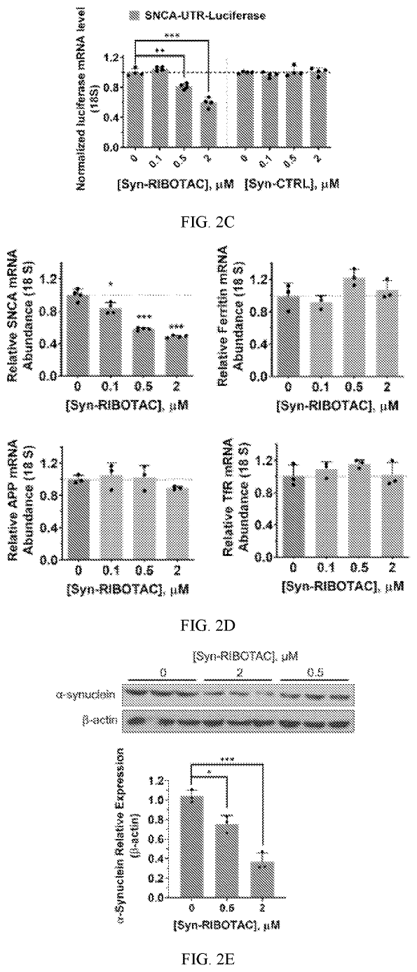

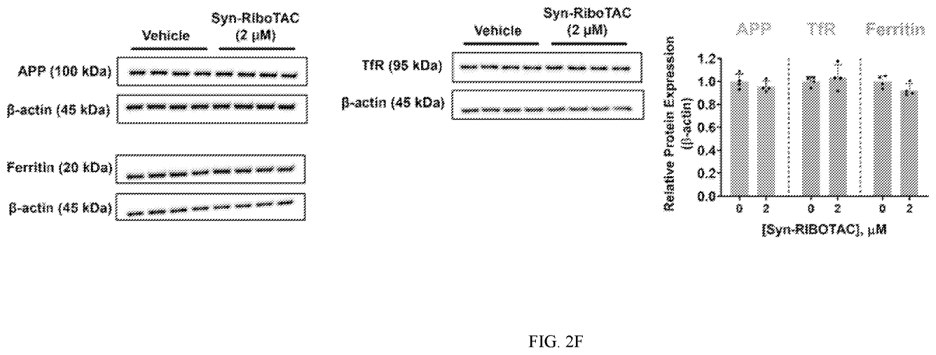

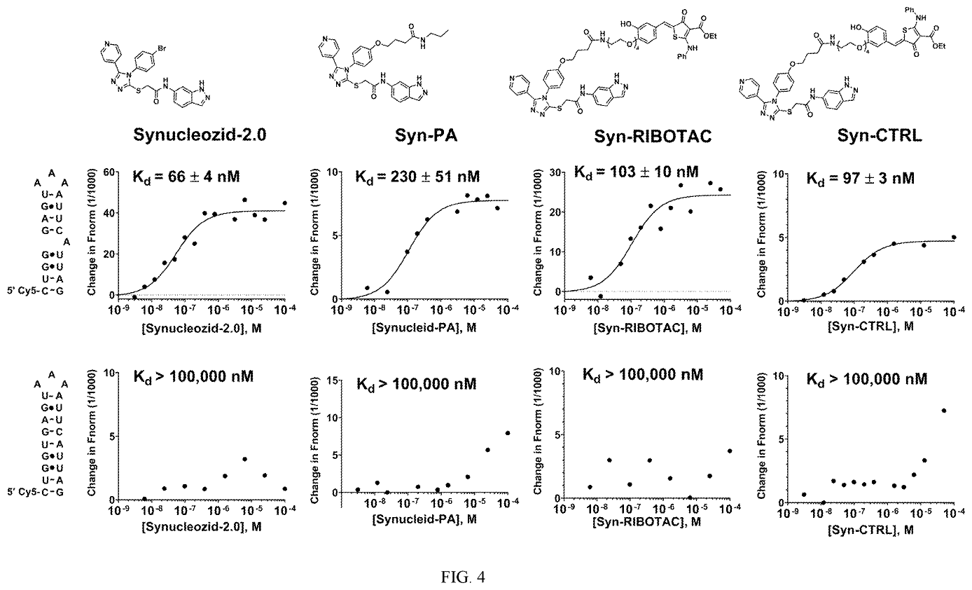

Therefore Synucleozid 2.0 was converted into its Syn-RiboTAC by conjugation to a heterocycle small molecule recruiting and activating RNase L. A control compound, Syn-CTRL (FIG. 2A), was also synthesized in which the RNase L-recruiting module was replaced with a regioisomer that is ~20-fold less active in its ability induce RNase L cleavage.⁴⁸ To confirm that the modification of the bromide did not interfere with the binding to the target, the binding affinities of Synucleozid 2.0 (K_{d}=66±4 nM), Syn-PA (replacing bromine with propyl amide; K_{d}=230±51 nM), Syn-RiboTAC(K_{d}=103±10 nM) and Syn-CTRL (K_{d}=97±3 nM) for the SNCA IRE were measured by microscale thermophoresis (MST). Notably, no significant change in K_{d} was observed for these derivatives of Synucleozid-2.0 and they retain the selectivity as no saturable binding was observed towards the fully base paired RNA (FIG. 4).

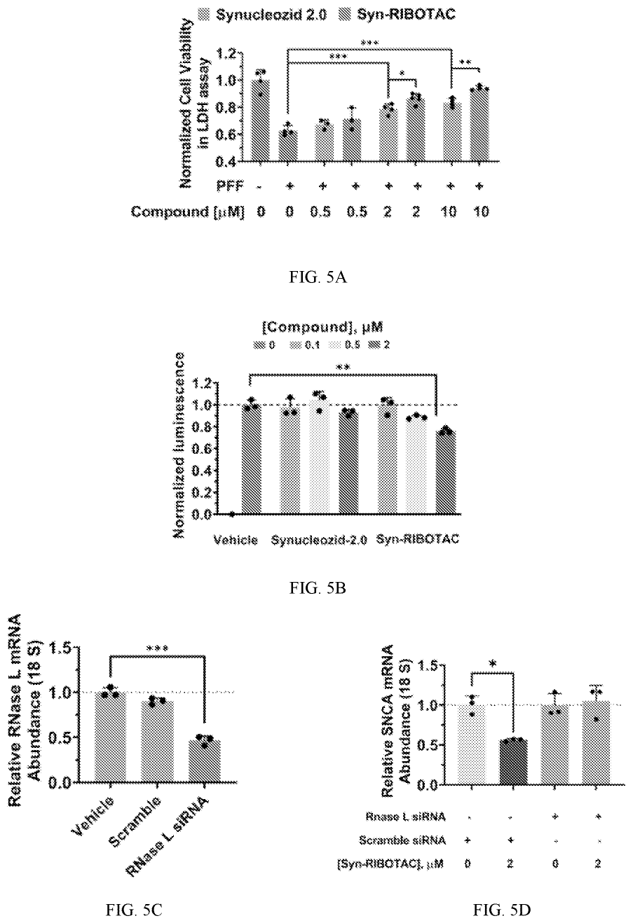

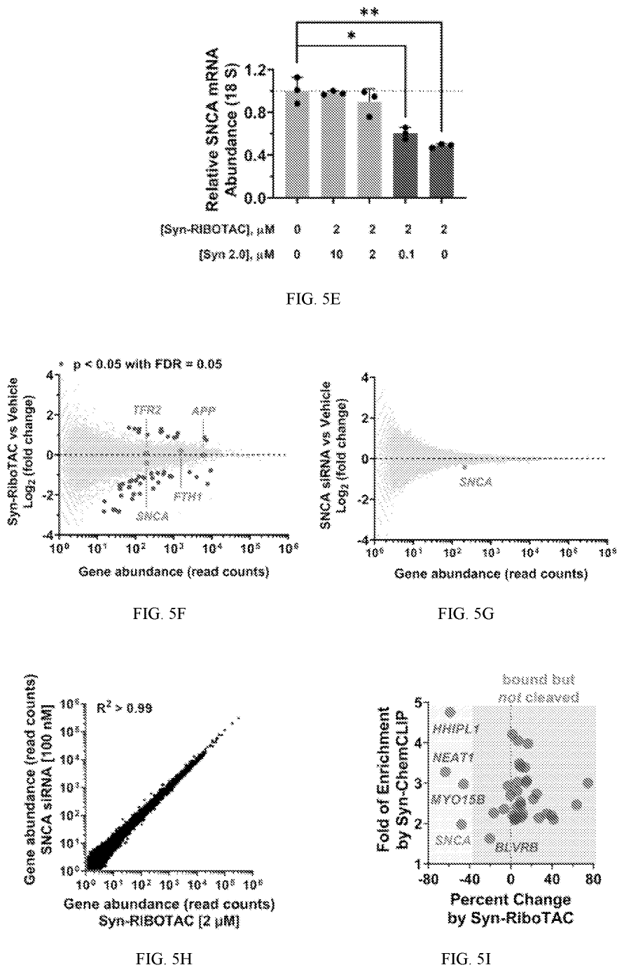

In the SNCA-UTR luciferase reporter assay, Syn-RiboTAC dose-dependently decreased the luminescence signal up to ~40% at 2 μM, to a greater extent than Synucleozid-2.0 (~25% decrease at 2 μM). Syn-CTRL only showed modest decrease at the highest concentration tested. This observed activity could be due to its reduced ability to induce cleavage or inhibiting ribosomal assembly as a binder (same mode of action as Synucleozid-2.0; FIG. 2B). Neither compound showed inhibition in the control luciferase reporter lacking an IRE. To provide support that inhibition of translation was due to cleavage of the RNA, the abundance of the SNCA IRE-luciferase transcript was measured by RT-qPCR. Syn-RiboTAC dose-dependently reduced the transcript's levels, with an ~50% reduction at the 2 μM dose. Syn-CTRL had no effect (FIG. 5C), indicating that it inhibits translation through a binding mechanism.

It was next determined whether Syn-RiboTAC can cleave endogenous SNCA mRNA and reduce α-synuclein protein levels in SH-SY5Y cells. Notably, not all SNCA transcripts harbor the IRE; previous RNA-seq studies showed that two of the five SNCA variants contain the targeted IRE and that these two variants accounts for ~50% of all expressed SNCA mRNA species.²² Thus, a maximum reduction of ~50% at the mRNA level could be expected. Syn-RiboTAC dose-dependently decreased SNCA mRNA levels, by 48□2% at the maximum dose (2 μM; FIG. 2D). A dose dependent reduction of α-synuclein protein levels was also observed, by 63□9% upon treatment with 2 μM of Syn-RiboTAC(FIG. 2E; compared to 53□3% by Synucleozid-2.0 at same concentration). It has been shown that a reduction of α-synuclein protein levels by as little as 25% is therapeutically beneficial in a mouse model.⁵⁰

### Example 2: Syn-RiboTAC Confers Cytoprotection to SH-SY5Y Cells Upon PFF Challenge

To study the cytoprotective effects of Syn-RiboTAC, the same PFF challenge assay that was used for Synucleozid-2.0 was completed in SH-SY5Y cells. Syn-RiboTAC dose dependently protected SH-SY5Y cells from cell death upon PFF challenge, with 86□4% of cells viable at the 2 μM dose and 94□2% at 10 μM. The cytoprotective effects conferred by Syn-RiboTAC are greater than that for Synucleozid-2.0, with a similar protection afforded by 2 μM of the RiboTAC(79□4% of cells viable) as afforded by 10 μM of the binding compound (83□3%) (FIG. 5A). Syn-RiboTAC also had longer lasting effects than Synucleozid-2.0, as demonstrated by a wash-out experiment using the luciferase reporter assay. While Syn-RiboTAC was still active 16 h after removal from the cells (24□12% inhibition as compared to 34□2% without the wash-out), Synucleozid-2.0 was inactive (FIG. 5B).

Syn-RiboTAC's activity is RNase L-dependent. To validate mode of action, RNase L was knocked down in SH-SY5Y cells with a pool of siRNAs (FIG. 5C), followed by treatment with Syn-RiboTAC. Knocking down RNase L levels ablated the effect of Syn-RiboTAC on SNCA mRNA, supporting that compound activity is RNase L-dependent (FIG. 5D). Syn-RiboTAC and Synucleozid-2.0 were validated to engage the same binding site in cells by a competition experiment using a constant concentration of Syn-RiboTAC(2 μM) and varying concentrations of Synucleozid-2.0 (0.1-10 μM). A dose-dependent restoration of SNCA mRNA levels were observed as a function of Synucleozid-2.0, suggesting that the two compounds compete for the same A bulge binding pocket of SNCA IRE (FIG. 5E).

### Example 3: Syn-RiboTAC is Selective Transcriptome- and Proteome-Wide

A previous analysis²² indicates that the SNCA IRE's A bulge and its closing base pairs is found only five times, in miR-1207 (Dicer cleavage site), miR-4310 (non-functional site), and in three tRNAs, amongst all human RNAs with known structures (n=7,436 structural elements). To investigate the selectivity of both Syn-RiboTAC and Synucleozid-2.0, all transcripts occupied by the small molecule were defined, and the effect of occupancy was then characterized transcriptome- and proteome-wide.

To gain initial insight into the selectivity of Syn-RiboTAC at the transcript level, the mRNA levels of other transcripts with IREs were measured in SH-SY5Y cells, including APP, ferritin, and TfR, by RT-qPCR. None of the three transcripts was affected by Syn-RiboTAC as they lack the same A bulge as SNCA mRNA (FIG. 2D). Similarly, no effect was observed on the protein levels by Syn-RiboTAC on APP, ferritin, and TfR, by Western blot (FIG. 5F). These results confirmed that Syn-RiboTAC retained the same specificity for the SNCA IRE that Synucleozid-2.0 did.

In unbiased transcriptome-wide studies using RNA-seq, among 30,175 genes detected, Syn-RiboTAC only affected 48 (0.16%) genes (log 2fold change >0.58 and p<0.05), with 34 genes downregulated and 14 genes upregulated (FIG. 5F). Both Syn-RiboTAC and an siRNA targeted to SNCA reduced abundance of the target, by ~35% and ~50%, respectively, although these reductions were not statistically significant by RNA-seq analysis. In accordance with their measurements by RT-qPCR, APP, ferritin, and TfR transcript levels were unaffected in the RNA-seq data (FIG. 5F). There are 690 other mRNAs that are known to contain IREs in the transcriptome.⁵¹ Of these, 447 were detectable in the RNA-seq data, only 19 of which were affected by Syn-RiboTAC treatment (11 downregulated and 8 upregulated), none of which harbor Syn-RiboTAC/Synucleozid-2.0's binding site in the SNCA IRE form. The transcriptome profile of Syn-RiboTAC treatment showed significant correlation with that of an siRNA targeted to SNCA with R²>0.99 (FIGS. 5G-5H). Collectively, these results suggest that Syn-RiboTAC is selective on the transcriptome with limited off-targets.

An unbiased proteome-wide analysis was conducted to assess selectivity further. At the proteome level, among 3,436 proteins detected, Syn-RiboTAC affected 194 (0.56%) proteins, with 114 proteins downregulated and 80 upregulated. Three proteins, MDH2, TCEB2, and RBM33, were upregulated by both compound treatments. Compared to previous proteomic profiling of Synucleozid 1.0 which affected 8% of global proteins,²² Syn-RiboTAC showed ~16-fold enhancement in selectivity on the proteome (FIG. 2A). By comparing RNA-seq and proteomics data sets, any direct correlation between changes in transcript and protein levels can be detected. Of the 194 proteins that showed changes upon treatment with Syn-RiboTAC, 86 (44%) also showed the corresponding change at the transcript level with modest global correlation (Person r=0.15, p<0.05).

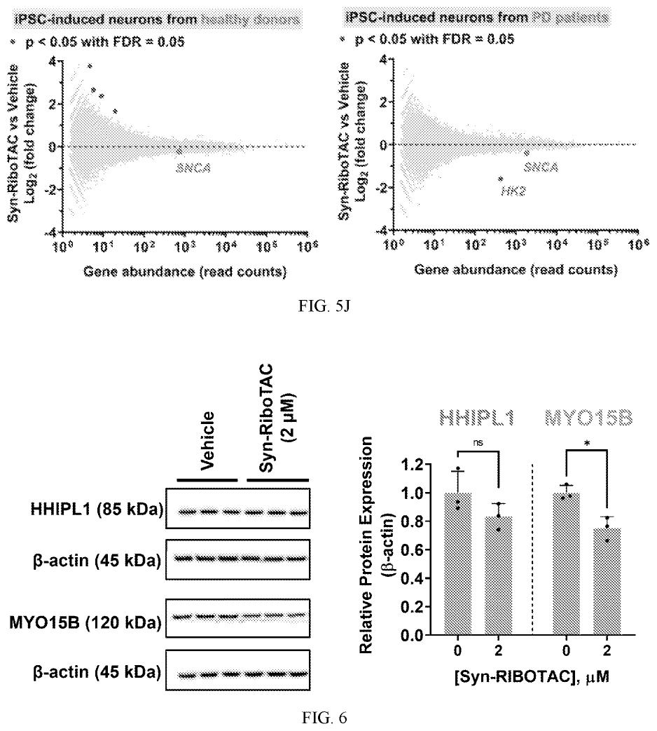

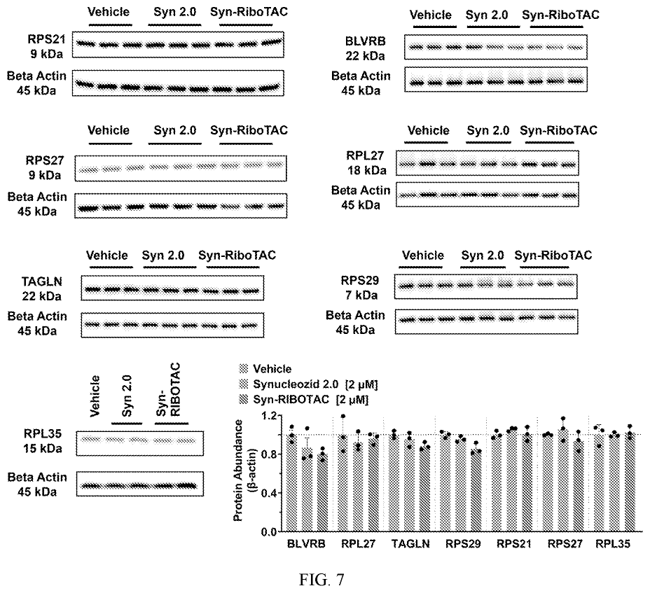

To determine if the changes observed in the transcriptome and proteome can be traced to direct target engagement, occupied targets were identified by Chem-CLIP and subsequent RNA-seq analysis. These studies revealed that Syn-CLIP enriched (bound) only 107 (0.6%) transcripts amongst 18,604 detected in SH-SY5Y cells. None of the 19 transcripts that contain are IRE and were reduced by Syn-RiboTAC were bound in Chem-CLIP studies. Of these 107 bound transcripts, 38 were detected in the RNA-seq analysis of Syn-RiboTAC; the levels of only four (including SNCA) were reduced (cleaved) by Syn-RiboTAC treatment (FIG. 5I). Of the three other transcripts, one (NEAT1) is a long noncoding (Inc)RNA while the other two (HHIPL1 and MYO15B) were not detectable in the proteomics data, whether vehicle- or RiboTAC-treated. By Western blotting, the levels of HHIPLI and MYO15B were only modestly reduced (~20%; FIG. 6).

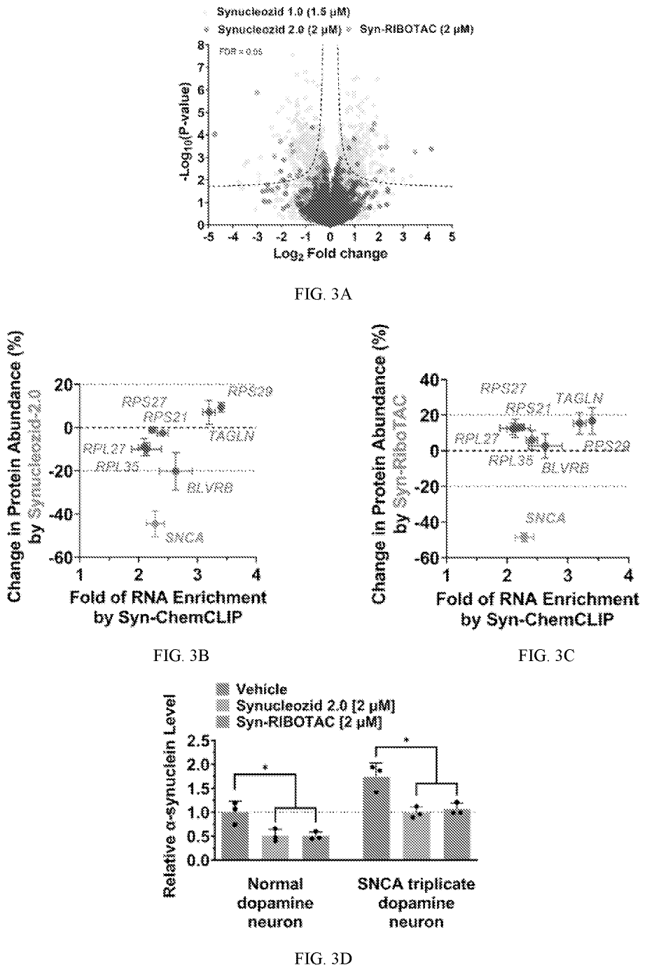

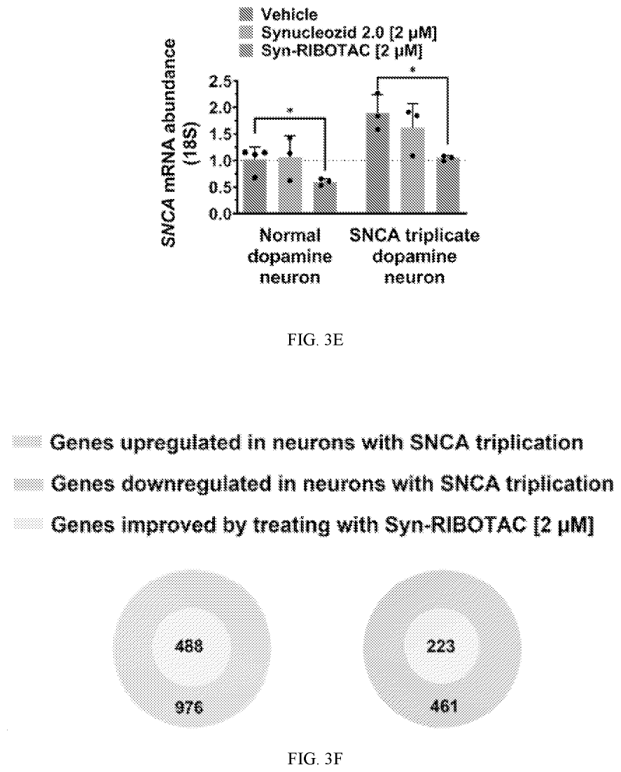

Of the remaining 34 transcripts bound by the small molecule and detectable in the RNA-seq data where their transcript levels were generally unaffected (BLVRB was reduced by ~20%, although this reduction was not statistically significant; FIG. 5I), the proteins encoded by seven of them were detectable by global proteomics. Their protein expression levels were not significantly altered by Synucleozid 2.0 (FIG. 3B) or Syn-RiboTAC treatment (FIG. 3C). It was verified that Synucleozid 2.0 and Syn-RiboTAC did not affect these seven proteins by Western blotting (FIG. 7).

### Example 4: Syn-RiboTAC Reduces α-Synuclein Protein Levels in Dopaminergic Neurons

Syn-RiboTAC was tested in human dopaminergic neurons as their death is a key hallmark of PD.⁵² Dopaminergic neurons were differentiated from induced pluripotent stem cells (iPSCs) donated by either a PD patient with SNCA triplication or a healthy individual. At 2 μM, Syn-RiboTAC decreased α-synuclein protein levels by ~50% (as determined by Western blotting), lowering the elevated levels of α-synuclein in patient-derived neurons to a level similar to that observed in healthy, untreated neurons (FIG. 3D). Synucleozid-2.0 had no effect on the SNCA mRNA levels while Syn-RiboTAC decreased transcript levels by ~50%, consistent with their respective mechanisms of action (FIG. 3E).

Total RNA-seq was performed to evaluate the global effects of Syn-RiboTAC in PD patient-derived neurons (FIG. 5J). Only 1 gene besides SNCA, hexokinase 2 (HK2) which does not harbor an IRE, is significantly decreased by Syn-RiboTAC treatment. HK2 is known to promote the apoptosis of dopaminergic neurons in PD and thus its decrease could alleviate PD pathologies.⁵³ Among 976 genes significantly upregulated in PD patient-derived neurons as compared to those derived from a healthy donor, 488 (50%) genes were improved by treating with Syn-RiboTAC; that is, Syn-RiboTAC decreased the levels of these genes. Similarly, among 461 genes significantly downregulated in PD patient-derived neurons, 223 (48%) genes were improved by treatment with Syn-RIBOTAC(FIG. 2F). Overall, Syn-RiboTAC rescued expression of about half of the genes deregulated in PD patient-derived neurons.

This disclosure presents an integrated approach to define drug-like chemical matter that binds to and affects RNA by targeted degradation, as applied to targeting the SNCA mRNA. Small molecule binding selectively inhibits translation of the mRNA, reducing levels of α-synuclein protein, a difficult-to-target IDP. Previously, both antibodies⁵⁴ and ASOs50 have been employed, the former to target extracellular α-synuclein, for therapeutic intervention of PD. One advantage of the small molecule approach presented herein is the potential for oral bioavailability and to target intracellular α-synuclein in both central and peripheral systems.

To meet many unmet medical needs, expanding druggability and developing the tools to do so can be beneficial. The present disclosure shows that an undruggable protein can be modulated by targeting its encoding mRNA, and that a streamlined workflow can be developed to define and study these compounds. This approach includes targeting structural elements throughout a mRNA. The observation that targeting RNA structural elements near a start codon has proven effective to modulate biology suggests they may be important, general targetable sites.

It has been demonstrated that one way to enhance the bioactivity while retaining the selectivity of small molecules targeting RNA is to convert simple RNA binders to RiboTAC degraders. RiboTACs do not require binding to functional sites to elicit activity as is the case for small molecules with binding mechanisms of action; rather, they can bind biologically inert sites and induce its degradation by endogenous nucleases. Furthermore, it is known that the most common way to elicit functional affects to eliminate disease causing RNA is by using oligonucleotide modalities, many of which has achieved approval. However, these large molecular weight compounds can have limited tissue distribution and are injected medicines. The approach herein for targeted RNA degradation using heterobifunctional molecules could be further advanced to provide molecules with broader tissue distribution that also could be developed into orally bioactive medicines.

### REFERENCES

1 Clamp, M. et al. Distinguishing protein-coding and noncoding genes in the human genome. Proc Natl Acad Sci USA 104, 19428-19433, doi: 10.1073/pnas.0709013104 (2007).
2 Hopkins, A. L. & Groom, C. R. The druggable genome. Nat Rev Drug Discov 1, 727-730, doi: 10.1038/nrd892 (2002).
3 Burslem, G. M. & Crews, C. M. Proteolysis-Targeting Chimeras as Therapeutics and Tools for Biological Discovery. Cell 181, 102-114, doi: 10.1016/j.cell.2019.11.031 (2020).
4 Lai, A. C. & Crews, C. M. Induced protein degradation: an emerging drug discovery paradigm. Nat Rev Drug Discov 16, 101-114, doi: 10.1038/nrd.2016.211 (2017).
5 Sakamoto, K. M. et al. Protacs: chimeric molecules that target proteins to the Skp1-Cullin-F box complex for ubiquitination and degradation. Proc Natl Acad Sci USA 98, 8554-8559, doi: 10.1073/pnas.141230798 (2001).
6 Sakamoto, K. M. et al. Development of Protacs to target cancer-promoting proteins for ubiquitination and degradation. Mol Cell Proteomics 2, 1350-1358 (2003).
7 Kostic, M. & Jones, L. H. Critical Assessment of Targeted Protein Degradation as a Research Tool and Pharmacological Modality. Trends in pharmacological sciences 41, 305-317, doi: 10.1016/j.tips.2020.02.006 (2020).
8 Ostrem, J. M. & Shokat, K. M. Direct small-molecule inhibitors of KRAS: from structural insights to mechanism-based design. Nat Rev Drug Discov 15, 771-785, doi: 10.1038/nrd.2016.139 (2016).
9 Rudolph, J. & Stokoe, D. Selective inhibition of mutant Ras protein through covalent binding. Angew Chem Int Ed Engl 53, 3777-3779, doi: 10.1002/anie.201400233 (2014).
10 Kamerkar, S. et al. Exosomes facilitate therapeutic targeting of oncogenic KRAS in pancreatic cancer. Nature 546, 498-503, doi: 10.1038/nature22341 (2017).
11 Lanman, B. A. et al. Discovery of a Covalent Inhibitor of KRAS(G12C)(AMG 510) for the Treatment of Solid Tumors. J Med Chem 63, 52-65, doi: 10.1021/acs.jmedchem.9b01180 (2020).
12 Counihan, J. L., Ford, B. & Nomura, D. K. Mapping proteome-wide interactions of reactive chemicals using chemoproteomic platforms. Curr Opin Chem Biol 30, 68-76, doi: 10.1016/j.cbpa.2015.11.007 (2016).
13 Schreiber, S. L. et al. Advancing Biological Understanding and Therapeutics Discovery with Small-Molecule Probes. Cell 161, 1252-1265, doi: 10.1016/j.cell.2015.05.023 (2015).
14 Lee, V. M. & Trojanowski, J. Q. Mechanisms of Parkinson's disease linked to pathological alpha-synuclein: new targets for drug discovery. Neuron 52, 33-38, doi: 10.1016/j.neuron.2006.09.026 (2006).
15 Luk, K. C. et al. Pathological alpha-synuclein transmission initiates Parkinson-like neurodegeneration in nontransgenic mice. Science 338, 949-953, doi: 10.1126/science.1227157 (2012).
16 Singleton, A. B. et al. alpha-Synuclein locus triplication causes Parkinson's disease. Science 302, 841, doi: 10.1126/science.1090278 (2003).
17 Maraganore, D. M. Rationale for therapeutic silencing of alpha-synuclein in Parkinson's disease. Journal of movement disorders 4, 1-7, doi: 10.14802/jmd.11001 (2011).
18 Ross, C. A. & Poirier, M. A. Protein aggregation and neurodegenerative disease. Nat Med 10 Suppl, S10-17, doi: 10.1038/nm1066 (2004).
19 Naylor, M. R., Bockus, A. T., Blanco, M. J. & Lokey, R. S. Cyclic peptide natural products chart the frontier of oral bioavailability in the pursuit of undruggable targets. Curr Opin Chem Biol 38, 141-147, doi: 10.1016/j.cbpa.2017.04.012 (2017).
20 Disney, M. D. Targeting RNA with small molecules to capture opportunities at the intersection of chemistry, biology, and medicine. J Am Chem Soc 141, 6776-6790 (2019).
21 Costales, M. G., Childs-Disney, J. L., Haniff, H. S. & Disney, M. D. How We Think about Targeting RNA with Small Molecules. J Med Chem, doi: 10.1021/acs.jmedchem.9b01927 (2020).
22 Zhang, P. et al. Translation of the intrinsically disordered protein alpha-synuclein is inhibited by a small molecule targeting its structured mRNA. Proc Natl Acad Sci USA 117, 1457-1467, doi: 10.1073/pnas. 1905057117 (2020).
23 Friedlich, A. L., Tanzi, R. E. & Rogers, J. T. The 5′-untranslated region of Parkinson's disease alpha-synuclein messengerRNA contains a predicted iron responsive element. Mol Psychiatry 12, 222-223, doi: 10.1038/sj.mp.4001937 (2007).
24 Zhou, Z. D. & Tan, E. K. Iron regulatory protein (IRP)-iron responsive element (IRE) signaling pathway in human neurodegenerative diseases. Mol Neurodegener 12, 75, doi: 10.1186/s13024-017-0218-4 (2017).
25 Wager, T. T., Hou, X., Verhoest, P. R. & Villalobos, A. Moving beyond rules: the development of a central nervous system multiparameter optimization (CNS MPO) approach to enable alignment of druglike properties. ACS chemical neuroscience 1, 435-449, doi: 10.1021/cn100008c (2010).
26 Disney, M. D. et al. Inforna 2.0: A Platform for the Sequence-Based Design of Small Molecules Targeting Structured RNAs. ACS Chem Biol 11, 1720-1728, doi: 10.1021/acschembio.6b00001 (2016).
27 Velagapudi, S. P. et al. Approved anti-cancer drugs target oncogenic non-coding RNAs. Cell chemical biology 25, 1086-1094.e1087, doi: 10.1016/j.chembiol.2018.05.015 (2018).
28 Haniff, H. S., Graves, A. & Disney, M. D. Selective small molecule recognition of RNA base pairs. ACS combinatorial science 20, 482-491, doi: 10.1021/acscombsci.8b00049 (2018).
29 Velagapudi, S. P., Gallo, S. M. & Disney, M. D. Sequence-based design of bioactive small molecules that target precursor microRNAs. Nat Chem Biol 10, 291-297, doi: 10.1038/nchembio.1452 (2014).
30 Velagapudi, S. P. et al. Design of a small molecule against an oncogenic noncoding RNA. Proc Natl Acad Sci USA 113, 5898-5903, doi: 10.1073/pnas.1523975113 (2016).
31 Costales, M. G. et al. Small molecule inhibition of microRNA-210 reprograms an oncogenic hypoxic circuit. J Am Chem Soc 139, 3446-3455, doi: 10.1021/jacs.6b11273 (2017).
32 Costales, M. G. et al. A designed small molecule inhibitor of a non-coding RNA sensitizes HER2 negative cancers to Herceptin. J Am Chem Soc 141, 2960-2974, doi: 10.1021/jacs.8b10558 (2019).
33 Xicoy, H., Wieringa, B. & Martens, G. J. The SH-SY5Y cell line in Parkinson's disease research: a systematic review. Mol Neurodegener 12, 10, doi: 10.1186/s13024-017-0149-0 (2017).
34 Eisenstein, R. S. Iron regulatory proteins and the molecular control of mammalian iron metabolism. Annual review of nutrition 20, 627-662, doi: 10.1146/annurev.nutr.20.1.627 (2000).
35 Volpicelli-Daley, L. A. et al. Exogenous alpha-synuclein fibrils induce Lewy body pathology leading to synaptic dysfunction and neuron death. Neuron 72, 57-71, doi: 10.1016/j.neuron.2011.08.033 (2011).
36 Cho, H. H. et al. Selective translational control of the Alzheimer amyloid precursor protein transcript by iron regulatory protein-1. J Biol Chem 285, 31217-31232, doi: 10.1074/jbc.M110.149161 (2010).
37 Rupani, D. N. & Connell, G. J. Transferrin receptor mRNA interactions contributing to iron homeostasis. Rna 22, 1271-1282, doi: 10.1261/rna.056184.116 (2016).
38 Jean, J. M. & Hall, K. B. 2-Aminopurine fluorescence quenching and lifetimes: role of base stacking. Proc Natl Acad Sci USA 98, 37-41 (2001).
39 Kaul, M., Barbieri, C. M. & Pilch, D. S. Fluorescence-based approach for detecting and characterizing antibiotic-induced conformational changes in ribosomal RNA: comparing aminoglycoside binding to prokaryotic and eukaryotic ribosomal RNA sequences. J Am Chem Soc 126, 3447-3453 (2004).
40 Shandrick, S. et al. Monitoring molecular recognition of the ribosomal decoding site. Angew Chem Int Ed Engl 43, 3177-3182 (2004).
41 Zarrinkar, P. P. & Williamson, J. R. Kinetic intermediates in RNA folding. Science 265, 918-924 (1994).
42 Velagapudi, S. P., Li, Y. & Disney, M. D. A cross-linking approach to map small molecule-RNA binding sites in cells. Bioorg Med Chem Lett 29, 1532-1536, doi: 10.1016/j.bmcl.2019.04.001 (2019).
43 Brittenham, G. M. et al. Efficacy of deferoxamine in preventing complications of iron overload in patients with thalassemia major. N Engl J Med 331, 567-573, doi: 10.1056/NEJM199409013310902 (1994).
44 Guo, B., Phillips, J. D., Yu, Y. & Leibold, E. A. Iron regulates the intracellular degradation of iron regulatory protein 2 by the proteasome. J Biol Chem 270, 21645-21651, doi: 10.1074/jbc.270.37.21645 (1995).
45 Chasse, H., Boulben, S., Costache, V., Cormier, P. & Morales, J. Analysis of translation using polysome profiling. Nucleic Acids Res 45, e15, doi: 10.1093/nar/gkw907 (2017).
46 Pena, C., Hurt, E. & Panse, V. G. Eukaryotic ribosome assembly, transport and quality control. Nat Struct Mol Biol 24, 689-699, doi: 10.1038/nsmb.3454 (2017).
47 Wager, T. T., Hou, X., Verhoest, P. R. & Villalobos, A. Central Nervous System Multiparameter Optimization Desirability: Application in Drug Discovery. ACS chemical neuroscience 7, 767-775, doi: 10.1021/acschemneuro.6b00029 (2016).
48 Costales, M. G. et al. Small-molecule targeted recruitment of a nuclease to cleave an oncogenic RNA in a mouse model of metastatic cancer. Proc Natl Acad Sci USA 117, 2406-2411, doi: 10.1073/pnas. 1914286117 (2020).
49 Zhang, P. et al. Reprogramming of Protein-Targeted Small-Molecule Medicines to RNA by Ribonuclease Recruitment. J Am Chem Soc 143, 13044-13055, doi: 10.1021/jacs.1c02248 (2021).
50 Alarcon-Aris, D. et al. Anti-alpha-synuclein ASO delivered to monoamine neurons prevents alpha-synuclein accumulation in a Parkinson's disease-like mouse model and in monkeys. EBioMedicine 59, 102944, doi: 10.1016/j.ebiom.2020.102944 (2020).
51 Baird, S. D., Turcotte, M., Korneluk, R. G. & Holcik, M. Searching for IRES. RNA 12, 1755-1785, doi: 10.1261/rna. 157806 (2006).
52 Naoi, M. & Maruyama, W. Cell death of dopamine neurons in aging and Parkinson's disease. Mech Ageing Dev 111, 175-188, doi: 10.1016/s0047-6374 (99) 00064-0 (1999).
53 Li, J. et al. Upregulated hexokinase 2 expression induces the apoptosis of dopaminergic neurons by promoting lactate production in Parkinson's disease. Neurobiol Dis 163, 105605, doi: 10.1016/j.nbd.2021.105605 (2022).
54 Weihofen, A. et al. Development of an aggregate-selective, human-derived alpha-synuclein antibody BIIB054 that ameliorates disease phenotypes in Parkinson's disease models. Neurobiology of Disease 124, 276-288, doi: 10.1016/j.nbd.2018.10.016 (2019).
55 Howe, J. A. et al. Selective small-molecule inhibition of an RNA structural element. Nature 526, 672-677, doi: 10.1038/nature15542 (2015).
56 Serganov, A., Polonskaia, A., Phan, A. T., Breaker, R. R. & Patel, D. J. Structural basis for gene regulation by a thiamine pyrophosphate-sensing riboswitch. Nature 441, 1167-1171, (2006).
57 O'Leary, C. A. et al. RNA structural analysis of the MYC mRNA reveals conserved motifs that affect gene expression. PLOS One 14, e0213758, doi: 10.1371/journal.pone.0213758 (2019).
58 Chen, J. L. et al. The RNA encoding the microtubule-associated protein tau has extensive structure that affects its biology. PLOS One 14, e0219210, doi: 10.1371/journal.pone.0219210 (2019).

### Example 5: Methods

General. The RNA oligonucleotides, including unlabeled, fluorescence-labeled, and 2-AP substituted RNA, were purchased from Dharmacon with HPLC purification by the manufacturer. All DNA oligonucleotides, including primers, gapmer antisense oligonucleotides (ASOs), were purchased from Integrated DNA Technologies. The DNAs were prepared by dissolving in nuclease free water and used without further purification. The siRNA pool targeting α-synuclein (ON-TARGETplus Human SNCA siRNA-SMARTpool) and the scramble siRNA control (ON-TARGETplus Non-targeting Pool) were ordered from Dharmacon. The siRNA pool targeting RNase L was purchased from Santa Cruz Biotechnology (SC-45965).

### In Vitro Methods.

Binding Affinity Measurements: Microscale Thermophoresis (MST). Binding affinity measurements were performed on a Monolith NT.115 MST system (NanoTemper Technologies) with Cy5-labeled oligonucleotides. Briefly, Cy5-labeled RNA (5 nM) was prepared in 1× Folding Buffer and folded by heating at 60° C. for 5 min and then slowly cooling to room temperature. After cooling, Tween-20 was added to a final concentration of 0.1% (v/v). Compound solutions were prepared separately in 30 μL of 1× Assay Buffer at a final concentration of 5 μM (1% (v/v) DMSO), followed by 1:1 serial dilutions in 1× Assay Buffer. RNA and compound solutions were then mixed 1:1 by volume.

Samples were incubated for 20 min at room temperature and then loaded into premium capillaries (NanoTemper Technologies, Cat #MO-K025). The following parameters were used for MST measurement: 5-20% LED power (adjusted to keep fluorescence intensity between 8000 and 18000), 80% MST power, Laser-On time=30 s, and Laser-Off time=5 s. The resulting data were analyzed by calculating the change in thermophoresis as a function of compound concentration and fitted by Equation (3) in MST analysis software (NanoTemper Technologies) to yield the dissociation constant (K_{d}):

$$f ( c ) = unbound + \frac{bound - unbound}{2} * ( F + c + {K}_{d} - sqrt ( {( F + c + {K}_{d} )}^{2} - 4 * F * c ) ) \qquad Eq . ( 3 )$$

where F is the concentration of fluorescently labeled RNA; unbound and bound refer to the thermophoresis signal at completely unbound and bound state of RNA, respectively; c is the concentration of the compound; f (c) is the thermophoresis signal at compound concentration of c; K_{d} is the dissociation constant.

### Cellular Methods

Cell culture. Human neuroblastoma SH-SY5Y cells (ATCC) were cultured in Dulbecco's Modified Eagle's Medium/F-12 1:1 mix medium (DMEM/F-12, GE Healthcare) supplemented with 10% fetal bovine serum (FBS, Atlanta Biologicals) and 1% Penicillin/Streptomycin (Gibco). HeLa cells (ATCC) were cultured in DMEM supplemented with 10% FBS and 1% GlutaMax (Gibco) and 1% Penicillin/Streptomycin. All cells were maintained in a humidified incubator at 37° C. and 5% CO₂. Cells were seeded in 100 mm dishes, 6-well, 12-well or 96-well plates until they reached ~60% confluency. Cells were then treated by replacing the growth medium with fresh medium containing the compound of interest or DMSO(vehicle; 0.1% v/v final concentration). Cells were tested for mycoplasma contamination prior to experiments (PromoKine, PK-CA91-1024).

RNA extraction and reverse transcription-quantitative polymerase chain reaction (RT-qPCR). Total RNA was extracted from cells using a Quick-RNA MiniPrep Kit (Zymo Research) per the manufacturer's protocol including DNase treatment. For each sample, 200 ng of RNA was reverse transcribed into cDNA by using a qScript cDNA Synthesis Kit (QuantaBio) according to the manufacturer's instructions. To assess the relative mRNA levels, RT-qPCR was performed in triplicate using SYBR Green PCR mater mix (Applied Biosystems) with a Quant Studio 5 Real-Time PCR system. Primers are provided in Table S2. The relative levels of mRNA were calculated by using the ΔΔCt method as previously described.⁸

Western blotting. After 48 h, cells were gently washed with ice-cold 1× DPBS, and total protein was extracted with Mammalian Protein Extraction Reagent (M-PER, Thermo Scientific) per the manufacturer's protocol. After quantifying protein concentration by using BCA Protein Assay Kit (Pierce Biotechnology), ~30 μg of total protein for each sample was separated using a 10% SDS-polyacrylamide gel and transferred onto a PVDF membrane. The membrane was fixed with 0.4% paraformaldehyde (pH=7.0) for 30 min at room temperature⁹ and then blocked with 5% (w/v) non-fat dry milk in 1× TBST (1× TBS containing 0.1% Tween-20) for 1 h at room temperature. The membrane was incubated with primary antibody of α-synuclein (1:1000, 610787 from BD Transduction Laboratories), Amyloid Precursor Protein (1:2000, ab32136 from Abcam), Ferritin (1:2000, ab75973 from Abcam), transferrin receptor (1:2000, ab84036 from Abcam), Transgelin (TAGLN, 1:1000, 10493-1-AP from Proteintech), RPS29 (1:1000, 17374-1-AP from Proteintech), RPL35 (1:1000, 14826-1-AP from Proteintech), BLVRB (1:1000, 17727-1-AP from Proteintech), RPS21 (1:1000, 16946-1-AP from Proteintech), RPS27 (1:1000, 15355-1-AP from Proteintech), RPL27 (1:1000, 14980-1-AP from Proteintech), HHIPL1 (1:1000, ABIN786053 from Antibodies-online Inc), or MYO15 (1:1000, ABIN2772840 from Antibodies-online Inc) in 1× TBST containing 5% (w/v) non-fat dry milk at 4° C. overnight. After washing 10 min each with 1× TBST three times, the membrane was incubated anti-mouse (7076s, Cell Signaling Technology) or anti-rabbit IgG (7074s, Cell Signaling Technology) horseradish peroxidase secondary antibody conjugate at room temperature for 1 h. After washing 10 min each with 1× TBST three times, immunocomplex signals of each protein were detected by using a SuperSignal West Pico Chemiluminescent Substrate (Pierce Biotechnology) according to the manufacturer's protocol. The memebrane was then stripped by using 1× Stripping Buffer (200 mM glycine, pH 2.2, 4 mM SDS, 1% (v/v) Tween 20) at room temperature for 15 min, followed by blocking with 5% (w/v) non-fat dry milk at room temperature for 1 h. β-actin was used to normalize the levels of all proteins, measured by incubating with a primary antibody for β-actin (1:5000, 3700S from Cell Signaling Technology) at 4° C. overnight. The secondary antibody incubation and imaging were the same as described above for other protein targets. Expression levels of protein in each sample were determined based on band intensity, quantified by ImageJ software, and then normalized to band intensity of β-actin.

Plasmid constructs and establishment of reporter gene overexpressing cells. Construction of pIRES-Luc-EGFP-puro plasmid and pCDH-α-syn-5′-UTR-Luc-EGFP-puro plasmid used in 5′ UTR-luciferase-based selectivity assay were previously described.⁴ Briefly, In-Fusion PCR cloning system (Takara Bio, Mountain View, CA, USA) was used for all plasmid constructions per the manufacturer's instructions. To ensure sequence accuracy, all plasmids generated in this study were confirmed by sequencing.

Luciferase assay. The effect of compounds on translation of SNCA IRE-containing transcript was measured by using a luciferase reporter assay. HeLa cells (~60% confluency) were transfected with plasmids (2 μg per 60 mm dish) by using JetPrime (Polyplus Transfection) according to the manufacturer's recommended procedures. Approximately 16 h after transfection, the transfection cocktail was removed, and the cells were trypsinized and seeded to 96-well plates (8,000 cells per well). After 6 h, cells were treated as described in Cell Culture. To measure the luciferase signals, the cells were then washed with 1× DPBS, and luciferase activity in each well was measured by using the ONE-Glo Luciferase Assay System (Promega) per the manufacturer's instructions. Luminescence from firefly luciferase reaction was measured using a Biomek FLx800 plate reader.

Lactate Dehydrogenase (LDH) assay. SH-SY5Y cells in 96-well plates (~60% confluency) were pre-treated with compounds in growth medium for 24 h, followed by addition of 50 ng/μL of human α-synuclein pre-formed fibrils (PFFs, SPR-322 from Eagle Biosciences). The cells were then incubated for an additional 48 h. Cell viability was measured using LDH (Lactate Dehydrogenase) Cytotoxicity Detection Kit (MK401; Takara) according to the manufacturer's instructions.

Cytotoxicity assay. SH-SY5Y cells were treated with compound for 48 h in growth medium. Cell viability and cytotoxicity were measured using CellTiter 96 Aqueous One Solution Cell Proliferation Assay (MTS) and LDH assay, respectively, according to the manufacturers' recommended instructions.

Transfection of siRNAs. The siRNAs were purchased from commercial sources as listed in General information. The α-synuclein siRNA (final concentration of 200 nM) or RNase L siRNA (final concentration of 50 nM) were transfected into SH-SY5Y cells (at ~60% confluency in 12-well plates) by using the Lipofectamine RNAiMax (Life technologies) per manufacturer's protocol.

RNA immunoprecipitation (IP). RNA IP was performed as previously described.⁴ Briefly, SH-SY5Y cells were seeded in 6-well plates (~60% confluency) and treated with vehicle (0.1% DMSO) or Synucleozid-2.0 (2 μM) as described above. After 48 h, the cells were washed with ice-cold 1× DPBS, harvested, and lysed for 20 min on ice in 100 μL of M-PER buffer supplemented with 1× Protease Inhibitor Cocktail III for Mammalian Cells (Research Products International Corp.) and 80 U RNaseOUT Recombinant Ribonuclease Inhibitor (Invitrogen). After centrifugation and collection of the supernatant, the supernatant was incubated with Dynabeads Protein A (Life Technologies) displaying primary antibodies for β-actin (Cell Signaling: 8H10D10), IRP-1 (Cell Signaling Technologies, catalog #D6S4J) or IRP-2 (Cell Signaling Technologies, catalog #D6E6W) overnight at 4° C. The beads were then washed three times with 1× DPBS containing 0.02% (v/v) Tween-20, followed by RNA extraction, cDNA synthesis, and RT-qPCR as described in RNA extraction and reverse transcription-quantitative PCR (RT-qPCR). Relative RNA expression in IRP fractions and β-actin fractions were determined by ΔΔCt method⁸ and normalized to 18S rRNA level as an endogenous reference housekeeping gene. Normalized fold change was calculated by dividing SNCA mRNA expression (relative to 18S) in the cDNA library prepared from RNA extracted from the IRP immunoprecipitated fractions by the relative SNCA mRNA expression (relative to 18S) in the cDNA library prepared from RNA extracted from the β-actin immunoprecipitated fractions as shown below in Eq. (3):

$$Normalized Fold Change = \frac{Relative RNA Expression in IRP fraction}{Relative RNA Expression in β - actin fraction} \qquad Eq . ( 3 )$$

Cellular Chem-CLIP. SHSY-5Y cells were seeded in 60 mm dishes and treated with Syn-ChemCLIP overnight. Cells were washed by 1× DPBS and irradiated by UV light (365 nm) in a UVP crosslinker (Analytik Jena, 40W) for 10 min. Total RNA was extracted as described above. Agarose-disulfide-azide beads (Click Chemistry Tools, 1238-2) were washed with 20 mM HEPES, pH 7.0, and mixed with 10 μg of input RNA. Sodium ascorbate (250 mM), CuSO₄ (10 mM), and THPTA (50 mM) were mixed at 1:1:1 ratio by volume and added to the tube with beads and RNA (45 μL per sample). The samples were rotated at 37° C. for 2 h, followed by brief centrifugation to decant the supernatant. Beads were washed six times with 1× Wash Buffer, and the RNA was recovered as described in the section above. The recovered RNA was directly subjected to RT-qPCR.

RNA-seq analysis. Total RNA was quantified using the Qubit 2.0 Fluorometer (Invitrogen) and an Agilent 2100 Bioanalyzer RNA nano chip. Samples with RIN>9.0 were proceeded to library preparation using NEBNext Ultra II Directional RNA Kit (Cat. #E7760, NEB) following manufacture's protocol. Briefly, 200 ng of total RNA was depleted of ribosomal RNA using the NEBNext rRNA Depletion Module. The product was chemically fragmented using NEBNext Magnesium RNA Fragmentation Module (E6150S, NEB) the at 94° C. for 15 min. First-strand synthesis was performed with random primers, and second strand was synthesized with dUTP in place of dTTP. After end repair and 3′ adenylation, the cDNA was ligated with adaptors and then treated with USER enzyme (Uracil-specific excision reagent). The product was PCR amplified with Illumina-barcoded primers to generate the cDNA libraries. The final libraries were analyzed on the bioanalyzer DNA chips, pooled in equimolar ratios, and loaded onto the NextSeq 500 v2.5 flow cell and sequenced with 2×40 bp paired-end chemistry. The raw data were aligned to the human genome using STAR.¹¹ Read counts were extracted using featureCounts,¹² and the differential gene analysis was performed using Deseq2.¹³

Treatment of SH-SY5Y cells with Syn-RiboTAC for proteome-wide studies. SH-SY5Y cells were seeded in 6-well plates (~60% confluency) and treated with vehicle (0.1% (v/v) DMSO) or Syn-RiboTAC as described above. After 48 h, the cells were gently washed twice with 1× DPBS, harvested by scraping, and resuspended in 1× DPBS. They were then lysed by sonication. Protein concentrations were determined by using BCA Protein Assay Kit (Pierce Biotechnology). Reduction of □-synuclein protein levels by Syn-RiboTAC were confirmed by Western blotting as described above. Protein samples were then denatured in 50 mM NH₄HCO₃, pH 8, containing 6 M urea and then reduced with 10 mM TCEP (tris(2-carboxyethyl)phosphine hydrochloride) for 30 min. The proteins were then alkylated with 25 mM iodoacetamide for 30 min in dark. Samples were diluted to 2 M urea containing 50 mM NH₄HCO₃, pH 8, and digested with trypsin (Thermo Scientific, 1.5 μL of 0.5 μg/μL) containing 1 mM CaCl₂) (100× stock in water) at 37° C. for 12 h. Samples were acidified by adding acetic acid to a final concentration of 5% (v/v), desalted by using a self-packed C18 spin column, and dried. Samples obtained were analyzed by LC-MS/MS(as described below), and the MS data were processed with MaxQuant (as described below).

LC-MS/MS analysis and MaxQuant analysis. Peptides obtained above were resuspended in water containing 0.1% (v/v) formic acid and analyzed by using EASY-nLC 1200 nano-UHPLC coupled to Q Exactive HF-X Quadrupole-Orbitrap mass spectrometer (Thermo Scientific). Parameters for the chromatography column and elution methods were the same as previously described.⁴ MS data were analyzed by using MaxQuant 14 (V1.6.1.0) and then searched against the human proteome (Uniprot) and a common list of contaminants (included in MaxQuant). The false discovery rate (FDR) for proteins, peptides and sites identification was set at 1%. Parameters were the same as described previously.⁴

Dopamine neuron induction. For dopamine neuronal induction,^{15,16} the dual-SMAD inhibition method¹⁷ was used for floor plate (FP)-based midbrain dopamine (DA) neuron induction with modifications. Prior to differentiation, Matrigel matrix (BD) was thawed at 4° C. overnight or on ice until it is dissolved. Matrigel coated 6-well plates were prepared according to manufacturer's instructions and incubated at 37° C. for at least 1 h before use. iPSCs were dissociated with Accutase (STEMCELL Technologies) and plated (3.5×10⁵-4×10⁵ cells per cm²) into a new Matrigel coated plate and grown in mTeSR medium (STEMCELL Technologies) with StemBeads FGF2 (StemCultures) and ROCK inhibitor Y27632 (5 μM, Tocris). After 24 to 48 h, when the cells reached 100% confluency, the differentiation protocol was initiated by feeding the cells with DMEM containing 15% KnockOut Serum Replacement (KSR, Gibco), 2 mM L-glutamine (L-Glut) and 10 μM β-mercaptoethanol for 11 days.

Floor Plate (FP) induction protocol was followed based on timed exposure to BMP inhibitor LDN193189 (100 nM, PeproTech), ALK inhibitor SB431542 (10 μM, Tocris), recombinant mouse sonic hedgehog N-terminus SHH C25II (100 ng/ml, R&D), Smoothened agonist Purmorphamine (2 μM, PeproTech) and GSK3 inhibitor CHIR99021 (CHIR; 3 μM, PeproTech). KSR medium was gradually shifted to N2 medium (Neurobasal/N2 with B27 Plus Supplement; Gibco) starting on day 5 of differentiation as described previously.¹⁸ On day 11, the medium was changed to Neurobasal/B27/L-Glut containing medium (NB/B27; Gibco) supplemented with CHIR (until day 13) and with BDNF (20 ng/ml; PeproTech), ascorbic acid (0.2 mM), GDNF (20 ng/ml; PeproTech), dibutyryl cAMP (0.5 mM; PeproTech), TGFβ3 (1 ng/ml; PeproTech), and γ-secretase inhibitor DAPT (10 μM; PeproTech) for 9 days. On Day 20 and Day 30 (midbrain DA neuron precursor stages), cells were dissociated using Accutase and replated at high cell density (8× 10⁵ cells per cm²) in dishes pre-coated with 15 μg/ml polyornithine/1 μg/ml laminin/2 μg/ml fibronectin in differentiation medium (NB/B27+BDNF, ascorbic acid, GDNF, dbcAMP, TGFβ3 and DAPT). Expression of dopamine (DA) neuron-specific and mature neuronal markers were confirmed by immunocytochemical staining for tyrosine hydroxylase (TH), LIM homeobox transcription factor 1a (Lmx1a), forkhead transcription Foxa2 and Nuclear receptor subfamily 4 group A member 2 (Nurr1). For testing the efficacy of compounds, mature DA neuron stages (Day 50 to 70) were used.

ELISA (Enzyme-Linked Immunosorbent Assay). Patient-derived iPSC cells were induced to dopamine neurons as described in Dopamine neuron induction. After compound treatment, total protein was extracted and quantified as described in Western blotting. The concentration of α-synuclein protein was measured by using Human Alpha-synuclein ELISA Kit (Abcam, ab260052) per the manufacturer's recommendations.

Quantification and statistical analysis. All data are reported as means with error bars representing S.D., unless noted otherwise. Statistical tests and number of replicates (n) are noted in each figure legend. Data were plotted a using GraphPad Prism 7 software, which was also used to calculate statistical significance.

### Example 6: Synthetic Methods and Characterization

Abbreviations. AcOH: Acetic acid. DCM: Dichloromethane. DIPEA: Diisopropyl ethyl amine. DMF: N, N-Dimethylformamide. DMSO: Dimethyl sulfoxide. EtOAc: Ethyl acetate. EtOH: Ethanol. HATU: Hexafluorophosphate azabenzotriazole tetramethyl uronium. HCl: Hydrogen chloride. HOAt, 3-hydroxytriazolo[4,5-b]pyridine. HPLC: High-performance liquid chromatography. MeOH: Methanol. NaOH: Sodium hydroxide. TFA: Trifluoro acetic acid.

General Synthetic Methods. Chemicals were purchased from the following companies: TCI, Alpha Aesar, CombiBlocks, and Sigma Aldrich. Pre-packed silica columns were purchased from Agela-Technologies. Preparative HPLC was performed using a Waters 1525 Binary HPLC pump equipped with a Waters 2487 dual absorbance (254 and 345 nm) detector system and a Waters Sunfire C18 column (5 μm, 19×150 mm). Compound purification was performed with a linear gradient from 0-100% methanol in water with 0.1% (v/v) TFA over 60 min (5 mL/min). Purity was assessed by analytical HPLC using a Waters Symmetry C18 column (5 μm, 4.6×150 mm) with a flow rate of 1 mL/min and a linear gradient from 0-100% methanol in water with 0.1% (v/v) TFA over 60 min. Mass spectra were recorded on an Applied Biosystems 4800 plus MALDI-TOF/TOF analyzer using an α-cyano-4-hydroxycinnamic acid matrix. 1H NMR spectra were collected on a Bruker 400 MHz NMR spectrometer and ¹³C NMR spectra were collected on Bruker 600 MHz NMR spectrometer. Chemical shifts are shown in ppm calculated relative to tetramethyl silane (TMS).

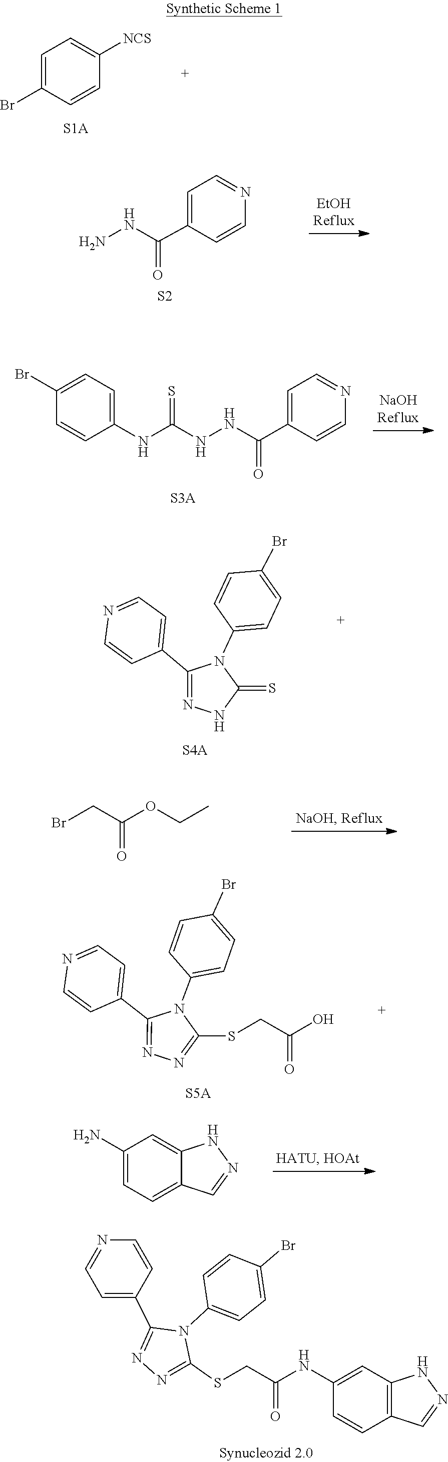

Compound S4A: A solution of S3A (synthesized as previously reported) 19 (1.5 g, 4.29 mmol) in 15 mL of 1 N NaOH aqueous solution was stirred at reflux for 2 h. After the reaction was cooled to room temperature, the solution was neutralized with AcOH to a pH of 5-6. The precipitates were collected by filtration and crystalized from EtOH to afford S4A as a white solid (1.25 g, 88%). ¹H NMR (400 MHz, DMSO-d⁶) δ (ppm) 8.67 (d, J=6.1 Hz, 2H), 7.81 (d, J=8.7 Hz, 2H), 7.47 (d, J=8.7 Hz, 2H), 7.34 (d, J=6.2 Hz, 2H). ¹³C NMR (600 MHz, DMSO-d⁶) δ (ppm) 169.2, 150.2, 148.4, 133.6, 133.2, 132.6, 130.9, 123.1, 122.1. HRMS(m/z): calculated for C₁₃H₁₀BrN₄S [M+H]⁺332.9804, found: 332.9766.

Compound S5A. A solution of S4A (664 mg, 2 mmol) and NaOH (240 mg, 6 mmol) in 6 mL of EtOH was stirred under reflux for 30 min. To the solution was added S5A (332 mg, 2 mmol). After stirring under reflux for another 4 h, the reaction mixture was cooled to room temperature and neutralized with 3 N HCl to pH 7. The precipitates were collected by filtration and washed with water to give S6 as a white solid (600 mg, 1.54 mmol, 77%). ¹H NMR (400 MHz, DMSO-d⁶) δ (ppm) 8.59 (d, J=6.1 Hz, 2H), 7.81 (d, J=8.7 Hz, 2H), 7.46 (d, J=8.7 Hz, 2H), 7.31 (d, J=6.2 Hz, 2H). ¹³C NMR (600 MHz, DMSO-d⁶) δ (ppm) 169.4, 153.1, 152.7, 150.7, 134.1, 133.7, 133.0, 130.2, 124.3, 122.2, 34.9. HRMS(m/z): calculated for C₁₅H₁₂BrN₄O₂S [M+H]⁺ 390.9859, found: 390.9835.

Synucleozid-2.0: A solution of S5A (130 mg, 0.33 mmol), S7 (53.2 mg, 0.4 mmol), HATU (190 mg, 0.5 mmol), HOAt (68 mg, 0.5 mmol) in 3 mL of DMF was stirred at room temperature overnight. The reaction mixture was purified by HPLC to give Synucleozid-2.0 as a TFA salt (75 mg, 0.148 mmol, 44%). ¹H NMR (600 MHz, DMSO-d⁶) δ (ppm) 10.52 (s, 1H), 8.65 (d, J=5.9 Hz, 2H), 8.09 (s, 1H), 7.97 (d, J=1 Hz, 1H), 7.82 (d, J=8.7 Hz, 2H), 7.68 (d, J=8.6 Hz, 2H), 7.51 (d, J=8.6 Hz, 2H), 7.41 (d, J=6.3 Hz, 2H), 7.08 (dd, J=8.7 Hz, 1.7 Hz), 4.29 (s, 2H). ¹³C NMR (600 MHz, DMSO-d⁶) δ (ppm) 165.5, 153.2, 152.0, 149.1, 140.3, 136.9, 134.9, 133.3, 132.5, 129.7, 123.9, 122.1, 120.8, 119.3, 113.9, 99.0, 37.1. HRMS(m/z): calculated for C₂₂H₁₇BrN₇OS [M+H]⁺ 506.0393, found: 506.0370.

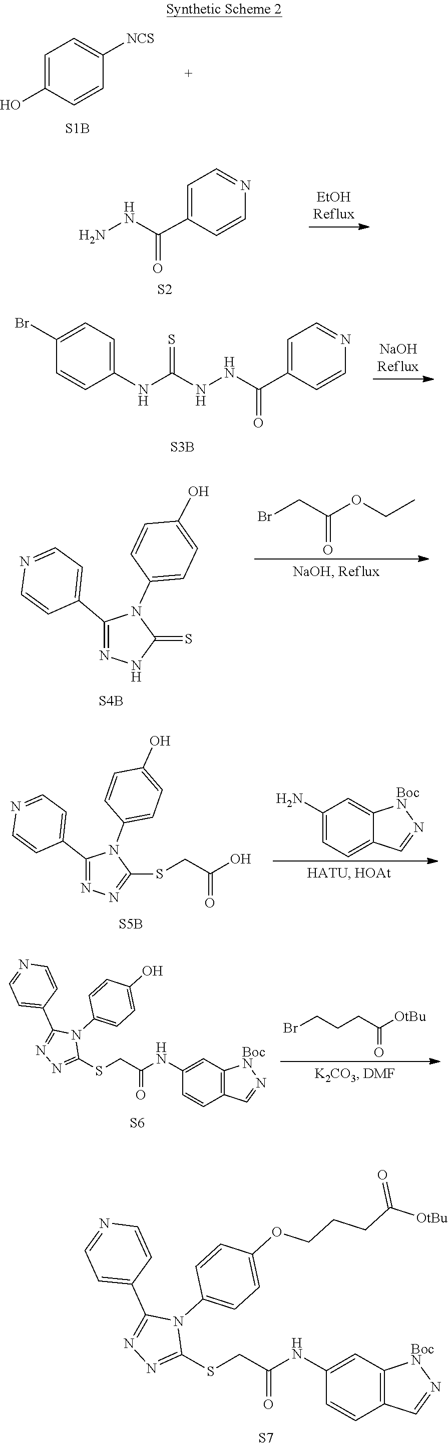

Compound S4B: The same procedure was followed as the synthesis of S4A described in Scheme 1 to afford S4B (890 mg, 3.3 mmol, 70%). ¹H NMR (400 MHz, DMSO-d⁶) δ (ppm) 9.98 (s, 1H) 8.57 (d, J=6.1 Hz, 2H), 7.26 (d, J=8.7 Hz, 2H), 7.18 (d, J=8.7 Hz, 2H), 7.34 (d, J-6.2 Hz, 2H). ¹³C NMR (600 MHz, DMSO-d⁶) δ (ppm) 170.0, 158.8, 150.6, 149.0, 133.8, 130.2, 125.6, 122.2, 116.5.

Compound S5B: The same procedure was followed as the synthesis of S5A described in Scheme 1 to afford S5B (480 mg, 1.5 mmol, 46%). ¹H NMR (400 MHz, DMSO-d⁶) δ (ppm) 10.18 (s, 1H) 8.58 (d, J=6.1 Hz, 2H), 7.31 (d, J=8.7 Hz, 2H), 7.26 (d, J=8.7 Hz, 2H), 6.92 (d, J=6.2 Hz, 2H), 4.07 (s, 2H). ¹³C NMR (600 MHz, DMSO-d⁶) δ (ppm) 169.7, 158.3, 152.8, 151.7, 149.5, 133.3, 128.2, 123.4, 120.7, 116.0, 33.4.

Compound S6: The same procedure was followed as the synthesis of Synucleozid 2.0 described in Scheme 1 to afford S6 (230 mg, 0.43 mmol, 57%). ¹H NMR (400 MHz, DMSO-d⁶) δ (ppm) 10.73 (s, 1H), 8.71 (s, 1H), 8.63 (d, J=5.7 Hz, 2H), 8.32 (s, 1H), 7.80 (d, J=8.6 Hz, 2H), 7.40 (m, 3H), 7.31 (d, J=8.6 Hz, 2H), 6.93 (d, J=8.7 Hz, 2H), 4.29 (s, 1H), 1.64 (s, 9H). ¹³C NMR (600 MHz, DMSO-d⁶) δ (ppm) 170.0, 158.8, 150.6, 149.0, 133.8, 130.2, 125.6, 122.2, 116.5.

Compound S7: To a solution of S6 (54 mg, 0.1 mmol) in DMF (0.5 mL) was added K₂CO₃ (69 mg, 0.5 mmol) followed by tert-butyl-4-bromobutanoate (44.6 mg, 0.2 mmol). The reaction was stirred at room temperature overnight and purified by HPLC to afford the product (49 mg, 0.072 mmol, 72%). ¹H NMR (400 MHz, DMSO-d⁶) δ (ppm) 8.7 (m, 3H), 8.22 (d, J=0.8 Hz, 1H), 8.77 (m, 3H), 7.40 (m, 3H), 7.11 (m, 2H), 4.22 (d, J=4.4 Hz, 2H), 4.06 (t, J=6.2 Hz, 2H), 2.44 (t, J=7.3 Hz, 2H), 2.06 (m, 1H), 1.72 (s, 7H), 1.46 (s, 9H). ¹³C NMR (600 MHz, DMSO-d⁶) δ (ppm) 174.3, 168.1, 162.3, 150.4, 146.8, 141.1, 130.0, 126.5, 124.6, 123.7, 122.9, 117.2, 105.9, 86.6, 81.7, 68.6, 32.8, 28.4, 25.7.

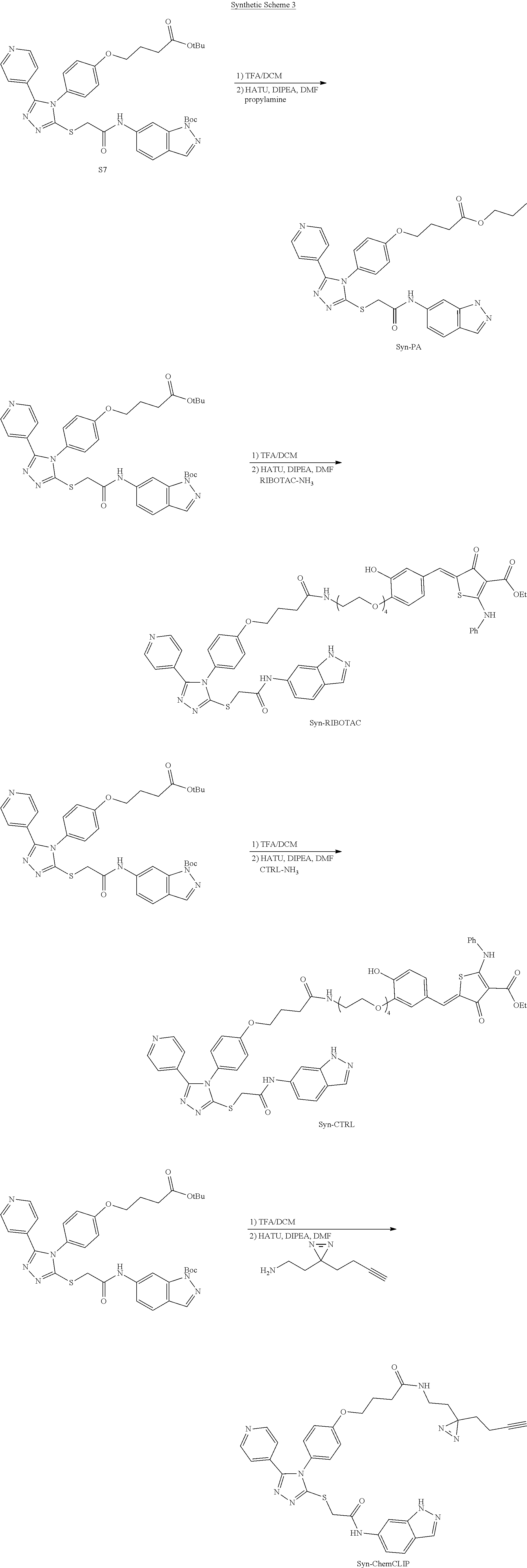

The same general method for all reactions in this scheme. Specifically, 3.4 mg of S7 (0.005 mmol) was dissolved in 100 μL of 1:1 TFA/DCM and stirred at room temperature for 1 hour. The solvent was evaporated and the solid was washed with Et₂O twice, followed by resuspension in 50 μL of DMF. HATU (0.01 mmol) and DIPEA (0.01 mmol) was added to the solution, followed by 0.02 mmol of the amine. The reaction was stirred at room temperature overnight and purified by HPLC. Syn-PA (yield 46%) HRMS(m/z) calculated for C₃₄H₃₇N₇O₆S [M+H]⁺: 572.2062, found: 572.2239. Syn-RIBOTAC(yield 34%) HRMS(m/z) calculated for C₅₄H₅₅N₉O₁₁S₂ [M+H]⁺: 1070.3522, found: 1070.3582. Syn-CTRL (yield 31%) HRMS(m/z) calculated for C₅₄H₅₅N₉O₁₁S₂ [M+H]⁺: 1070.3522, found: 1070.3552. Syn-ChemCLIP (yield 67%) HRMS(m/z) calculated for C₃₃H₃₂N₁₀O₃S: 649.2440, found: 649.2451.

### REFERENCES (Example 6)

1 Disney, M. D. et al. Inforna 2.0: A Platform for the Sequence-Based Design of Small Molecules Targeting Structured RNAs. ACS Chem Biol 11, 1720-1728, doi: 10.1021/acschembio.6b00001 (2016).
2 Velagapudi, S. P. et al. Approved Anti-cancer Drugs Target Oncogenic Non-coding RNAs. Cell Chem Biol 25, 1086-1094 e1087, doi: 10.1016/j.chembiol.2018.05.015 (2018).
3 Wager, T. T., Hou, X. J., Verhoest, P. R. & Villalobos, A. Moving beyond Rules: The Development of a Central Nervous System Multiparameter Optimization (CNS MPO) Approach To Enable Alignment of Druglike Properties. Acs Chem Neurosci 1, 435-449, doi: 10.1021/cn100008c (2010).
4 Zhang, P. et al. Translation of the intrinsically disordered protein alpha-synuclein is inhibited by a small molecule targeting its structured mRNA. Proc Natl Acad Sci USA 117, 1457-1467, doi: 10.1073/pnas. 1905057117 (2020).
5 Velagapudi, S. P. et al. Approved anti-cancer drugs target oncogenic non-coding RNAs. Cell chemical biology 25, 1086-1094.e1087, doi: 10.1016/j.chembiol.2018.05.015 (2018).
6 Haniff, H. S., Graves, A. & Disney, M. D. Selective small molecule recognition of RNA base pairs. ACS combinatorial science 20, 482-491, doi: 10.1021/acscombsci.8b00049 (2018).
7 McDowell, J. A. & Turner, D. H. Investigation of the structural basis for thermodynamic stabilities of tandem GU mismatches: solution structure of (rGAGGUCUC)2 by two-dimensional NMR and simulated annealing. Biochemistry 35, 14077-14089, doi: 10.1021/bi9615710 (1996).
8 Velagapudi, S. P. et al. Defining RNA-Small Molecule Affinity Landscapes Enables Design of a Small Molecule Inhibitor of an Oncogenic Noncoding RNA. ACS Cent. Sci. 3, 205-216, doi: 10.1021/acscentsci.7b00009 (2017).
9 Lee, B. R. & Kamitani, T. Improved immunodetection of endogenous alpha-synuclein. PLOS One 6, e23939, doi: 10.1371/journal.pone.0023939 (2011).
10 Yang, W. Y., Wilson, H. D., Velagapudi, S. P. & Disney, M. D. Inhibition of non-ATG translational events in cells via covalent small molecules targeting RNA. J Am Chem Soc 137, 5336-5345, doi: 10.1021/ja507448y (2015).
11 Dobin, A. et al. STAR: ultrafast universal RNA-seq aligner. Bioinformatics 29, 15-21, doi: 10.1093/bioinformatics/bts635 (2013).
12 Liao, Y., Smyth, G. K. & Shi, W. featureCounts: an efficient general purpose program for assigning sequence reads to genomic features. Bioinformatics 30, 923-930, doi: 10.1093/bioinformatics/btt656 (2014).
13 Love, M. I., Huber, W. & Anders, S. Moderated estimation of fold change and dispersion for RNA-seq data with DESeq2. Genome Biol 15, doi: ARTN 55010.1186/s13059-014-0550-8 (2014).
14 Cox, J. & Mann, M. MaxQuant enables high peptide identification rates, individualized p.p.b.-range mass accuracies and proteome-wide protein quantification. Nat Biotechnol 26, 1367-1372, doi: 10.1038/nbt.1511 (2008).
15 Kriks, S. et al. Dopamine neurons derived from human ES cells efficiently engraft in animal models of Parkinson's disease. Nature 480, 547-551, doi: 10.1038/nature10648 (2011).

16 Oni, E. N. et al. Increased nicotine response in iPSC-derived human neurons carrying the CHRNA5 N₃₉₈ allele. Sci Rep 6, 34341, doi: 10.1038/srep34341 (2016).
17 Chambers, S. M. et al. Highly efficient neural conversion of human ES and iPS cells by dual inhibition of SMAD signaling (vol 27, pg 275, 2009). Nat Biotechnol 27, 485-485, doi: 10.1038/nbt0509-485a (2009).
18 Chambers, S. M. et al. Highly efficient neural conversion of human ES and iPS cells by dual inhibition of SMAD signaling. Nat Biotechnol 27, 275-280, doi: 10.1038/nbt. 1529 (2009).
19 Oruc, E. E., Rollas, S., Kandemirli, F., Shvets, N. & Dimoglo, A. S. 1,3,4-thiadiazole derivatives. Synthesis, structure elucidation, and structure-antituberculosis activity relationship investigation. J Med Chem 47, 6760-6767, doi: 10.1021/jm0495632 (2004).

TABLE 1
Sequences of other oligonucleotide sequences used in the present disclosure

| | | SEQ ID | | |
|---|---|---|---|---|
| | Sequence | NO: | Experiment | Supplier |
| SNCA | 5′-TTCAAGCCTTCTGCCTTTCCACCC | 1 | qPCR | IDT |
| Fwd | | | | |
| SNCA | 5′-TCTCAGCAGCAGCCACAACTCCCT | 2 | qPCR | IDT |
| Rev | | | | |
| β-actin | 5′-CATGTACGTTGCTATCCAGGC | 3 | qPCR | IDT |
| Fwd | | | | |
| β-actin | 5′-CTCCTTAATGTCACGCACGAT | 4 | qPCR | IDT |
| Rev | | | | |
| 18S | 5′-GTAACCCGTTGAACCCCATT | 5 | qPCR | IDT |
| Fwd | | | | |
| 18S | 5′-CCATCCAATCGGTAGTAGCG | 6 | qPCR | IDT |
| Rev | | | | |
| TfR | 5′-AGGAACCGAGTCTCCAGTGA | 7 | qPCR | IDT |
| Fwd | | | | |
| TfR | 5′-ATCAACTATGATCACCGAGT | 8 | qPCR | IDT |
| Rev | | | | |
| APP | 5′-TGGCCAACATGATTAGTGAACC | 9 | qPCR | IDT |
| Fwd | | | | |
| APP | 5′-AAGATGGCATGAGAGCATCGT | 10 | qPCR | IDT |
| Rev | | | | |
| Ferritin | 5′-ACTGATGAAGCTGCAGAACC | 11 | qPCR | IDT |
| Fwd | | | | |
| Ferritin | 5′-GTCACCCAATTCTTTGATGG | 12 | qPCR | IDT |
| Rev | | | | |
| Firefly | 5′-GCCATGAAGCGCTACGCCCTGG | 13 | qPCR | IDT |
| Fwd | | | | |
| Firefly | 5′-TCTTGCTCACGAATACGACGGTGG | 14 | qPCR | IDT |
| Rev | | | | |
| Cy5- | 5′-Cy5-CUGGGAGUAAAAUUCAUUAG | 15 | in vitro | Dharmacon |
| WT-IRE | | | binding | |
| Cy5- | 5′-Cy5-CUGGUGAGUAAAAUUCAUUAG | 16 | in vitro | Dharmacon |
| BP-IRE | | | binding | |
| WT- | 5′- | 17 | AbsorbArray | Dharmacon |
| IRE | CUGGGAGUGGCCAUUCGACGACAGUG | | and In | |
| | UGGUGUAAAGGAAUUCAUUAG | | vitro Chem- | |
| | | | CLIP | |
| BP-IRE | 5′- | 18 | In vitro | Dharmacon |
| | CUGGUGAGUGGCCAUUCGACGCCACA | | Chem-CLIP | |
| | GUGUGGUGUCGAAUGGCCAUUCAUUA | | | |
| | G | | | |

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### Equivalents and Scope

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A compound of Formula (I):
B-L-R (I),
or a pharmaceutically acceptable salt, solvate, hydrate, polymorph, co-crystal, tautomer, stereoisomer, isotopically labeled compound, or prodrug thereof, wherein:
B is an RNA binder of Formula (II): 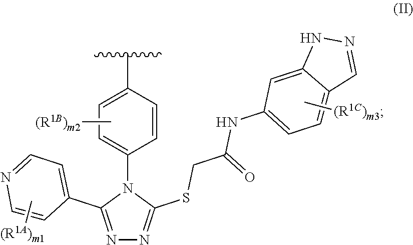
each occurrence of R^{1A}, R^{1B}, and R^{1C} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring,
each of m1, m2, and m3 is independently 0, 1, 2, 3, or 4;
L is a linker; and
R is an RNAse L recruiter.

2. The compound of claim 1, or a pharmaceutically acceptable salt, solvate, hydrate, polymorph, co-crystal, tautomer, stereoisomer, isotopically labeled compound, or prodrug thereof, wherein B is an RNA binder of Formula (II-a), Formula (II-b), or Formula (II-c):

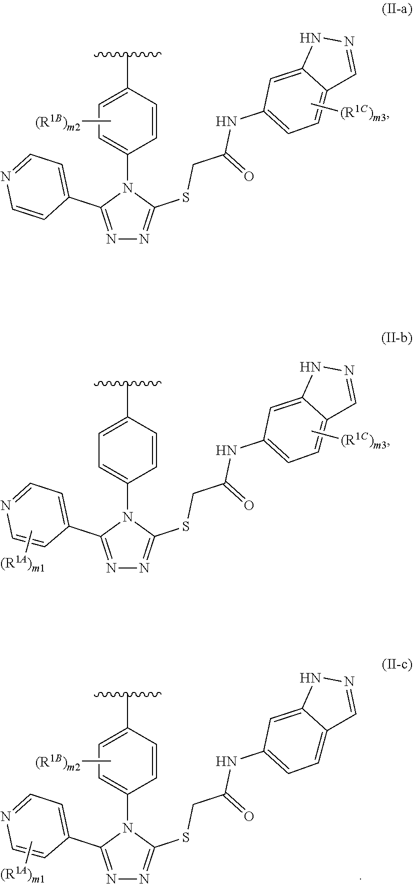

3. The compound of any one of claim 1 or 2, or a pharmaceutically acceptable salt, solvate, hydrate, polymorph, co-crystal, tautomer, stereoisomer, isotopically labeled compound, or prodrug thereof, wherein B is an RNA binder of Formula (II-d):

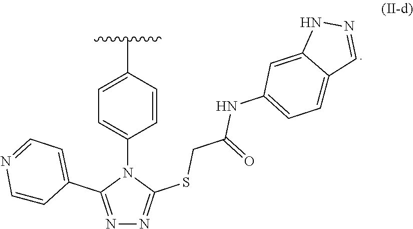 [SMILES: *c1ccc(-n2c(SCC(=O)Nc3ccc4cn[nH]c4c3)nnc2-c2ccncc2)cc1]

4. The compound of any one of claims 1-3, or a pharmaceutically acceptable salt, solvate, hydrate, polymorph, co-crystal, tautomer, stereoisomer, isotopically labeled compound, or prodrug thereof, wherein L is of Formula (III): 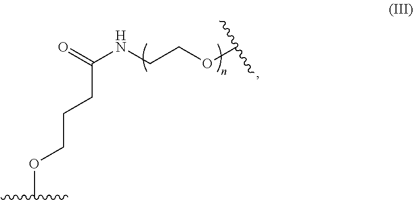
wherein n is 0, 1, 2, 3, 4, 5, 6, 7, 8, 9, or 10.

5. The compound of any one of claims 1-4, or a pharmaceutically acceptable salt, solvate, hydrate, polymorph, co-crystal, tautomer, stereoisomer, isotopically labeled compound, or prodrug thereof, wherein L is of formula:

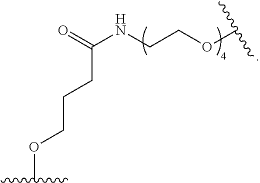

6. The compound of any one of claims 1-5, or a pharmaceutically acceptable salt, solvate, hydrate, polymorph, co-crystal, tautomer, stereoisomer, isotopically labeled compound, or prodrug thereof, wherein R is an RNAse L recruiter of Formulae (IV-a) or (IV-b):

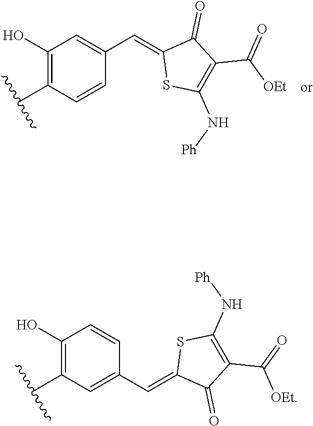 [SMILES: *c1ccc(/C=C2\SC(Nc3ccccc3)=C(C(=O)OCC)C2=O)cc1O ; *c1cc(/C=C2\SC(Nc3ccccc3)=C(C(=O)OCC)C2=O)ccc1O]

7. The compound of any one of claims 1-6, or a pharmaceutically acceptable salt, solvate, hydrate, polymorph, co-crystal, tautomer, stereoisomer, isotopically labeled compound, or prodrug thereof, wherein R is an RNAse L recruiter of Formula (IV-a):

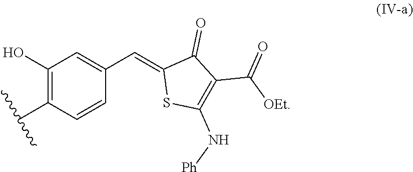 [SMILES: *c1ccc(/C=C2\SC(Nc3ccccc3)=C(C(=O)OCC)C2=O)cc1O]

8. The compound of any one of claims 1-4, 6, or 7, or a pharmaceutically acceptable salt, solvate, hydrate, polymorph, co-crystal, tautomer, stereoisomer, isotopically labeled compound, or prodrug thereof, wherein the compound is of Formula (I-a-i):

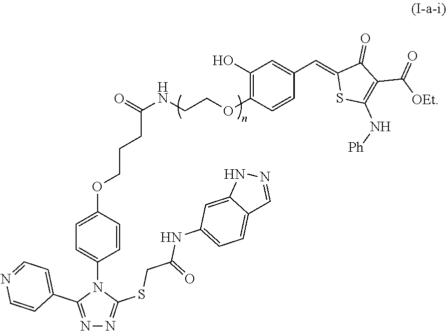

9. The compound of any one of claims 1-8, or a pharmaceutically acceptable salt, solvate, hydrate, polymorph, co-crystal, tautomer, stereoisomer, isotopically labeled compound, or prodrug thereof, wherein the compound is of formula:

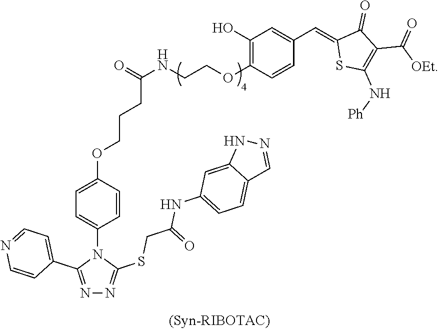

10. The compound of any one of claims 1-6, or a pharmaceutically acceptable salt, solvate, hydrate, polymorph, co-crystal, tautomer, stereoisomer, isotopically labeled compound, or prodrug thereof, wherein R is an RNAse L recruiter of Formula (IV-b):

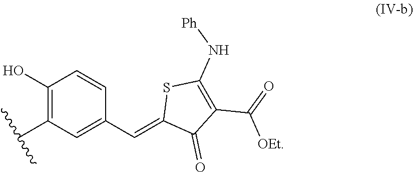 [SMILES: *c1cc(/C=C2\SC(Nc3ccccc3)=C(C(=O)OCC)C2=O)ccc1O]

11. The compound of any one of claims 1-4, 6, or 10, or a pharmaceutically acceptable salt, solvate, hydrate, polymorph, co-crystal, tautomer, stereoisomer, isotopically labeled compound, or prodrug thereof, wherein the compound is of Formula (I-b-i):

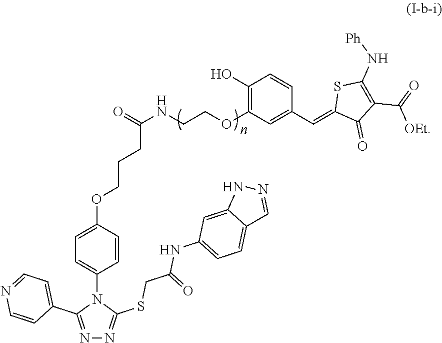

12. The compound of any one of claims 1-6, 10, or 11, or a pharmaceutically acceptable salt, solvate, hydrate, polymorph, co-crystal, tautomer, stereoisomer, isotopically labeled compound, or prodrug thereof, wherein the compound is of formula:

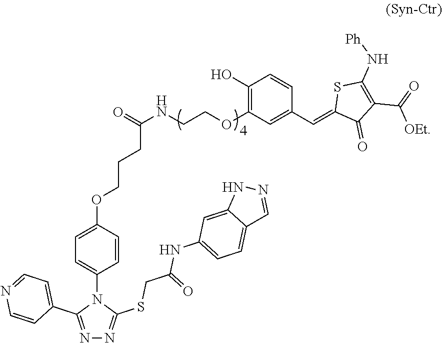

13. A compound of formula: 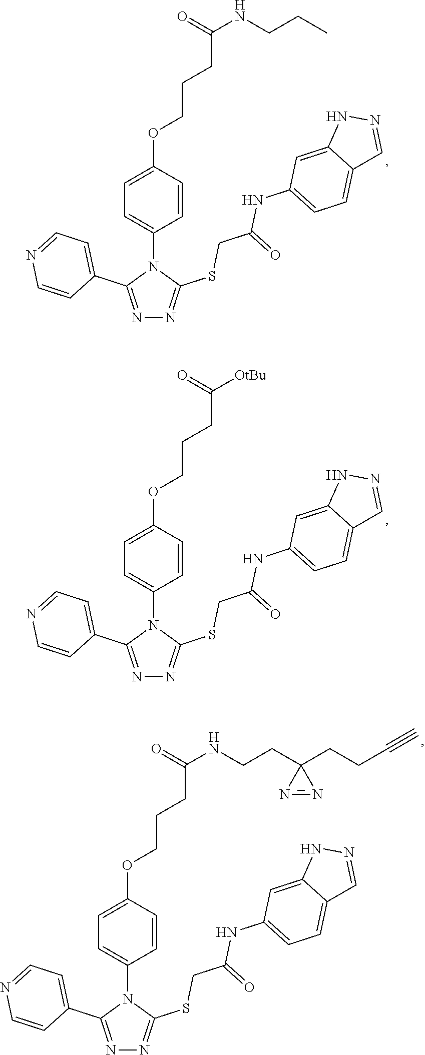 [SMILES: CCCNC(=O)CCCOc1ccc(-n2c(SCC(=O)Nc3ccc4cn[nH]c4c3)nnc2-c2ccncc2)cc1 ; CC(C)(C)OC(=O)CCCOc1ccc(-n2c(SCC(=O)Nc3ccc4cn[nH]c4c3)nnc2-c2ccncc2)cc1 ; C#CCCC1(CCNC(=O)CCCOc2ccc(-n3c(SCC(=O)Nc4ccc5cn[nH]c5c4)nnc3-c3ccncc3)cc2)N=N1]
or a pharmaceutically acceptable salt, solvate, hydrate, polymorph, co-crystal, tautomer, stereoisomer, isotopically labeled compound, or prodrug thereof.

14. A composition comprising the compound of any one of claims 1-13, or a pharmaceutically acceptable salt, solvate, hydrate, polymorph, co-crystal, tautomer, stereoisomer, isotopically labeled compound, or prodrug thereof, and an excipient.

15. A method of binding RNase L in a subject in need thereof or in a cell, tissue, or biological sample, comprising administering to the subject in need thereof or contacting the cell, tissue, or biological sample with an effective amount of:
a compound of any one of claims 1-13, or a pharmaceutically acceptable salt, solvate, hydrate, polymorph, co-crystal, tautomer, stereoisomer, isotopically labeled compound, or prodrug thereof; or
the composition of claim 14.

16. The method of claim 15, wherein binding RNase L comprises activating RNase L.

17. The method of any one of claim 15 or 16, wherein binding RNase L comprises inducing RNase L dimerization.

18. A method of binding an RNA target in a subject in need thereof or in a cell, tissue, or biological sample, comprising administering to the subject in need thereof or contacting the cell, tissue, or biological sample with an effective amount of:
a compound of any one of claims 1-13, or a pharmaceutically acceptable salt, solvate, hydrate, polymorph, co-crystal, tautomer, stereoisomer, isotopically labeled compound, or prodrug thereof; or
the composition of claim 14.

19. The compound of any one of claims 1-13, or a pharmaceutically acceptable salt, solvate, hydrate, polymorph, co-crystal, tautomer, stereoisomer, isotopically labeled compound, or prodrug thereof, or the composition of claim 14, for use in binding an RNA target in a subject in need thereof or in a cell, tissue, or biological sample.

20. The compound of any one of claims 1-13, or a pharmaceutically acceptable salt, solvate, hydrate, polymorph, co-crystal, tautomer, stereoisomer, isotopically labeled compound, or prodrug thereof, or the composition of claim 14, for use in the manufacture of a medicament for binding an RNA target in a subject in need thereof or in a cell, tissue, or biological sample.

21. A method of effecting degradation of an RNA target in a subject in need thereof or in a cell, tissue, or biological sample, comprising administering to the subject in need thereof or contacting the cell, tissue, or biological sample with an effective amount of:
a compound of any one of claims 1-13, or a pharmaceutically acceptable salt, solvate, hydrate, polymorph, co-crystal, tautomer, stereoisomer, isotopically labeled compound, or prodrug thereof; or
the composition of claim 14.

22. The compound of any one of claims 1-13, or a pharmaceutically acceptable salt, solvate, hydrate, polymorph, co-crystal, tautomer, stereoisomer, isotopically labeled compound, or prodrug thereof, or the composition of claim 14, for use in effecting degradation of an RNA target in a subject in need thereof or in a cell, tissue, or biological sample.

23. The compound of any one of claims 1-13, or a pharmaceutically acceptable salt, solvate, hydrate, polymorph, co-crystal, tautomer, stereoisomer, isotopically labeled compound, or prodrug thereof, or the composition of claim 14, for use in the manufacture of a medicament for effecting degradation of an RNA target in a subject in need thereof or in a cell, tissue, or biological sample.

24. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-23, further comprising recruiting RNase L.

25. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-24, further comprising cleaving the RNA target.

26. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-20, 24, and 25, further comprising effecting degradation of the RNA target.

27. The method, compound for use, pharmaceutically acceptable salt, solvate, hydrate, polymorph, co-crystal, tautomer, stereoisomer, isotopically labeled compound, or prodrug thereof for use, or composition for use of any one of claims 21-25, further comprising binding the RNA target.

28. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-27, wherein the RNA target is SNCA mRNA.

29. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-28, further comprising decreasing an amount of SNCA mRNA.

30. The method, compound for use, pharmaceutically acceptable salt, solvate, hydrate, polymorph, co-crystal, tautomer, stereoisomer, isotopically labeled compound, or prodrug thereof for use, or composition for use of claim 29, wherein the amount of SNCA mRNA is decreased by at least about 48%.

31. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-30, wherein the binding or effecting degradation is selective for SNCA mRNA compared to other mRNA.

32. The method, compound for use, pharmaceutically acceptable salt, solvate, hydrate, polymorph, co-crystal, tautomer, stereoisomer, isotopically labeled compound, or prodrug thereof for use, or composition for use of claim 31, wherein the binding or effecting degradation is selective for SNCA mRNA compared to APP mRNA, ferritin mRNA, and TfR mRNA.

33. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-32, wherein the compound, pharmaceutically acceptable salt, solvate, hydrate, polymorph, co-crystal, tautomer, stereoisomer, isotopically labeled compound, or prodrug thereof interacts with an SNCA mRNA iron-responsive element (IRE).

34. The method, compound for use, pharmaceutically acceptable salt, solvate, hydrate, polymorph, co-crystal, tautomer, stereoisomer, isotopically labeled compound, or prodrug thereof for use, or composition for use of claim 33, wherein the SNCA mRNA IRE is located in the SNCA mRNA 5′ untranslated region (5′ UTR).

35. The method, compound for use, pharmaceutically acceptable salt, solvate, hydrate, polymorph, co-crystal, tautomer, stereoisomer, isotopically labeled compound, or prodrug thereof for use, or composition for use of any one of claims 33 and 34, wherein the SNCA mRNA IRE comprises an A bulge binding pocket.

36. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-35, further comprising a cytoprotective effect.

37. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-36, further comprising decreasing an amount of hexokinase 2 (HK2).

38. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-37, further comprising decreasing an amount of α-synuclein.

39. The method, compound for use, pharmaceutically acceptable salt, solvate, hydrate, polymorph, co-crystal, tautomer, stereoisomer, isotopically labeled compound, or prodrug thereof for use, or composition for use of claim 38, wherein the amount of α-synuclein is decreased by at least about 63%.

40. The method, compound for use, pharmaceutically acceptable salt, solvate, hydrate, polymorph, co-crystal, tautomer, stereoisomer, isotopically labeled compound, or prodrug thereof for use, or composition for use of any one of claims 38 and 39, wherein the α-synuclein is decreased in Parkinson's Disease patient-derived dopaminergic neurons.

41. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-40, further comprising rescuing expression of genes abnormally expressed in Parkinson's Disease patient-derived dopaminergic neurons.

42. The method, compound for use, pharmaceutically acceptable salt, solvate, hydrate, polymorph, co-crystal, tautomer, stereoisomer, isotopically labeled compound, or prodrug thereof for use, or composition for use of any one of claims 18-41, wherein:
about 50% of genes significantly downregulated in Parkinson's Disease patient-derived dopaminergic neurons are increased; and/or
about 50% of genes significantly upregulated in Parkinson's Disease patient-derived dopaminergic neurons are decreased.

43. A method of treating or preventing a disease in a subject in need thereof, comprising administering to the subject in need thereof an effective amount of:
a compound of any one of claims 1-13, or a pharmaceutically acceptable salt, solvate, hydrate, polymorph, co-crystal, tautomer, stereoisomer, isotopically labeled compound, or prodrug thereof; or
the composition of claim 14.

44. The compound of any one of claims 1-13, or a pharmaceutically acceptable salt, solvate, hydrate, polymorph, co-crystal, tautomer, stereoisomer, isotopically labeled compound, or prodrug thereof, or the composition of claim 14, for use in treating a disease in a subject in need thereof.

45. The compound of any one of claims 1-13, or a pharmaceutically acceptable salt, solvate, hydrate, polymorph, co-crystal, tautomer, stereoisomer, isotopically labeled compound, or prodrug thereof, or the composition of claim 14, for use in the manufacture of a medicament for treatment of a disease in a subject in need thereof.

46. The method, compound for use, pharmaceutically acceptable salt, solvate, hydrate, polymorph, co-crystal, tautomer, stereoisomer, isotopically labeled compound, or prodrug thereof for use, or composition for use of any one of claims 43-45, wherein the disease is associated with SNCA mRNA.

47. The method, compound for use, pharmaceutically acceptable salt, solvate, hydrate, polymorph, co-crystal, tautomer, stereoisomer, isotopically labeled compound, or prodrug thereof for use, or composition for use of any one of claims 43-46, wherein the disease is a neurodegenerative disease.

48. The method, compound for use, pharmaceutically acceptable salt, solvate, hydrate, polymorph, co-crystal, tautomer, stereoisomer, isotopically labeled compound, or prodrug thereof for use, or composition for use of claim 47, wherein the neurodegenerative disease is Parkinson's Disease, Alzheimer's disease, Lewy bodies' disease, or Muscular System Atrophy.

49. The method, compound for use, pharmaceutically acceptable salt, solvate, hydrate, polymorph, co-crystal, tautomer, stereoisomer, isotopically labeled compound, or prodrug thereof for use, or composition for use of any one of claim 47 or 48, wherein the neurodegenerative disease is Parkinson's Disease.

50. A method of preparing a compound of Formulae (I-a) or (I-b): 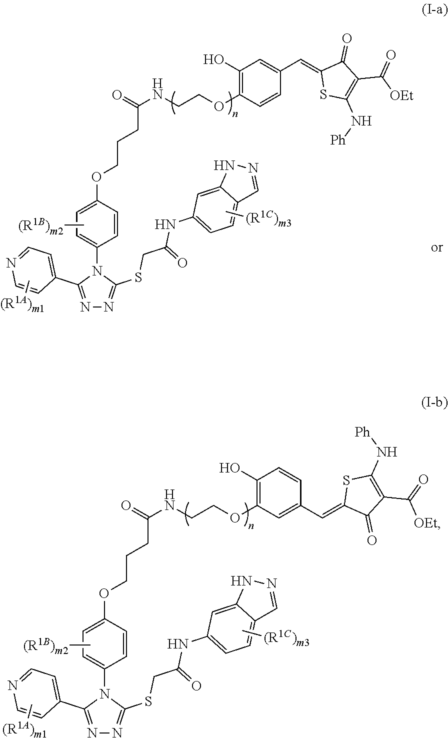
or a pharmaceutically acceptable salt, solvate, hydrate, polymorph, co-crystal, tautomer, stereoisomer, isotopically labeled compound, or prodrug thereof, comprising reacting a compound of Formulae (V-a) or (V-b): 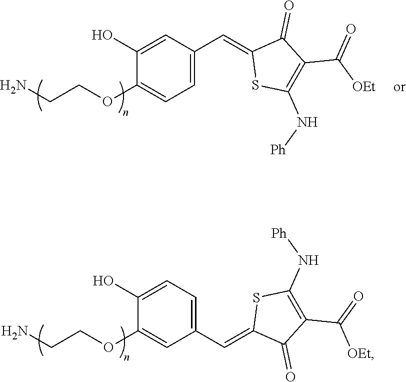
or a salt thereof, with a compound of Formula (VI): 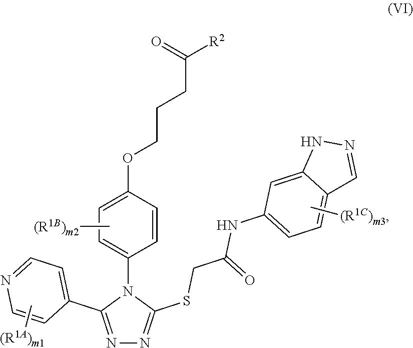
or a salt thereof, wherein:
R² is halogen or —OR^{2a};
R^{2a} is hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each occurrence of R^{1A}, R^{1B}, and R^{1C} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. each of m1, m2, and m3 is independently 0, 1, 2, 3, or 4; and
n is 0, 1, 2, 3, 4, 5, 6, 7, 8, 9, or 10.

51. The method of claim 50, wherein the compound of Formula (VI) is of Formula (VI-a): 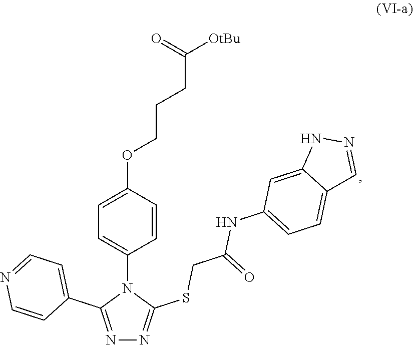 [SMILES: CC(C)(C)OC(=O)CCCOc1ccc(-n2c(SCC(=O)Nc3ccc4cn[nH]c4c3)nnc2-c2ccncc2)cc1]
or a salt thereof.

52. The method of any one of claims 50 and 51, further comprising alkylating a compound of Formula (VII): 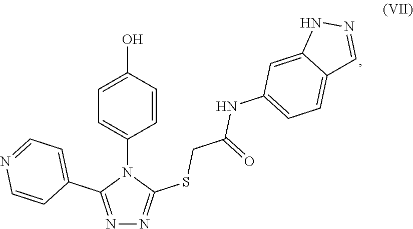 [SMILES: O=C(CSc1nnc(-c2ccncc2)n1-c1ccc(O)cc1)Nc1ccc2cn[nH]c2c1]
or a salt thereof, to provide the compound of Formula (VI), or salt thereof.

53. The method of any one of claims 50-52, further comprising alkylating a compound of formula: 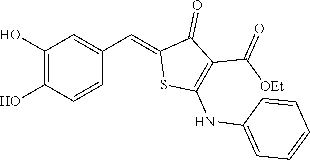 [SMILES: CCOC(=O)C1=C(Nc2ccccc2)S/C(=C\c2ccc(O)c(O)c2)C1=O]
or a salt thereof, to provide the compound of formula: 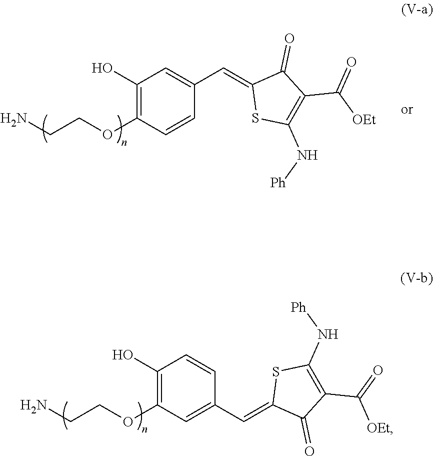
or a salt thereof.

54. A kit comprising the compound of any one of claims 1-13, or a pharmaceutically acceptable salt, solvate, hydrate, polymorph, co-crystal, tautomer, stereoisomer, isotopically labeled compound, or prodrug thereof, or the composition of claim 14, and instructions for its use.
